(19) United States
(12) Patent Application Publication
(10) Pub. No.: US 2008/0220429 A1
(43) Pub. Date: Sep. 11, 2008

(54) Single nucleotide polymorphisms and the identification of lactose intolerance

(76) Inventors: Sarah A. Tishkoff, Ellicott City, MD (US); Floyd Allan Reed, Plon (DE)

Correspondence Address: KRAMER & AMADO, P.C., 1725 DUKE STREET, SUITE 240, ALEXANDRIA, VA, 22314, US

(21) Appl. No.: 11/976,676

(22) Filed: Oct. 26, 2007

Related U.S. Application Data
(60) Provisional application No. 60/863,220, filed on Oct. 27, 2006.

(51) Int. Cl. C12Q 1/68; C12N 15/11; C12N 15/00; C12N 5/06

(52) U.S. Cl. 435/6; 536/23.2; 435/320.1; 435/325

(57) ABSTRACT

The present invention relates generally to methods, kits, genotyping and/or nucleic acid molecules associated with the identification of a predisposition for lactase persistence, lactase non-persistence, lactose tolerance and/or lactose intolerance. The methods of the present invention comprise in general determining the presence or absence of at least one variant allele having one or more single nucleotide polymorphisms within a gene associated with the expression of lactase-phlorizin hydrolase. The single nucleotide polymorphism is selected from the group consisting essentially of C-14010, G-13915 and G-13907, as measured from the start of the LCT gene.

This application claims priority to U.S. Provisional Patent Application 60/863,220, which was filed on Oct. 27, 2006, the contents of which are incorporated herein in their entirety.

### FIELD OF THE INVENTION

The present invention relates to single nucleotide polymorphisms associated with lactase persistence and non-persistence. The present invention also relates to methods for determining a predisposition for lactase persistence, lactase non-persistence, lactose tolerance and/or lactose intolerance. The present invention further relates to individual genotyping and/or nucleic acid molecules associated with lactase persistence, lactase non-persistence, lactose tolerance and/or lactose intolerance.

### BACKGROUND OF THE INVENTION

In most humans, the ability to digest lactose, the main carbohydrate present in milk, declines rapidly after weaning because of decreasing levels of the enzyme lactase-phlorizin hydrolase (LPH). LPH is predominantly expressed in the small intestine, where it hydrolyzes lactose into glucose and galactose, sugars that are easily absorbed into the bloodstream. See e.g., Swallow, D. M, Genetics of lactase persistence and lactose intolerance, Annu Rev Genet 37, 197-219 (2003). However, some individuals, particularly descendants from populations that have traditionally practiced cattle domestication, maintain the ability to digest milk and other dairy products into adulthood. These individuals have what is termed the “Lactase Persistence” (LP) trait. For example, the frequency of LP has been found to be high in Northern European populations (>90% in Swedes and Danes), decreasing in frequency across Southern Europe and the Middle East (˜50% in Spanish, French, and pastoralist Arabic populations), and is low in non-pastoralist Asian and African populations (˜1% in Chinese, ˜5-20% in West African agriculturalists). See e.g., Swallow, D. M., Genetics of lactase persistence and lactose intolerance, Annu Rev Genet 37, 197-219 (2003); Hollox, E. & Swallow, D. M., in The Genetic Basis of Common Diseases (eds. King, R. A., Rotter, J. I. & Motulsky, A. G.) 250-265 (Oxford University Press, Oxford, 2002); Durham, W. H., Coevolution: Genes, Culture, and Human Diversity (Stanford University Press, Stanford, 1992). However, LP has been found to be common in pastoralist populations from Africa (˜90% in Tutsi, ˜50% in Fulani). See e.g., Swallow, D. M., Genetics of lactase persistence and lactose intolerance, Annu Rev Genet 37, 197-219 (2003); Durham, W. H., Coevolution: Genes, Culture, and Human Diversity (Stanford University Press, Stanford, 1992).

LP is inherited as a Mendelian dominant trait in Europeans. See e.g., Swallow, D. M., Genetics of lactase persistence and lactose intolerance, Annu Rev Genet 37, 197-219 (2003), Hollox, E. & Swallow, D. M., in The Genetic Basis of Common Diseases (eds. King, R. A., Rotter, J. I. & Motulsky, A. G.) 250-265 (Oxford University Press, Oxford, 2002); Enattah, N. S. et al., Identification of a variant associated with adult-type hypolactasia, Nat Genet 30, 233-7 (2002). Adult expression of the gene coding for LPH (LCT), located on 2q21, is thought to be regulated by cis-acting elements, as illustrated by FIG. 1. Wang, Y et al., The lactase persistence/non-persistence polymorphism is controlled by a cis-acting element, Hum Mol Genet 4, 657-62 (1995). In one study, a linkage disequilibrium (LD) and haplotype analysis of Finnish pedigrees identified two single nucleotide polymorphisms (SNPs) associated with the LP trait: C/T-13910 and G/A-22018, located ˜14 kb and ˜22 kb upstream of LCT, respectively, within introns 9 and 13 of the adjacent minichromosome maintenance 6 (MCM6) gene. Enattah, N. S. et al., Identification of a variant associated with adult-type hypolactasia, Nat Genet 30, 233-7 (2002). The T-13910 and A-22018 alleles were 100% and 97%, respectively, associated with LP in the Finnish study, and moreover, the T-13910 allele was ˜86%-98% associated with LP in other European populations. See Poulter, M. et al., The causal element for the lactase persistence/non-persistence polymorphism is located in a 1 Mb region of linkage disequilibrium in Europeans, Ann Hum Genet 67, 298-311 (2003); Hogenauer, C. et al., Evaluation of a new DNA test compared with the lactose hydrogen breath test for the diagnosis of lactase non-persistence, Eur J Gastroenterol Hepatol 17, 371-6 (2005); Ridefelt, P. & Hakansson, L. D., Lactose intolerance: lactose tolerance test versus genotyping, Scand J Gastroenterol 40, 822-6 (2005). Although these alleles could have simply been in LD with an unknown regulatory mutation, several additional lines of evidence, including mRNA transcription studies in intestinal biopsy samples and reporter gene assays driven by the LCT promoter in vitro, indicate that the C/T-13910 SNP regulates LCT transcription in Europeans. See Kuokkanen, M. et al., Transcriptional regulation of the lactase-phlorizin hydrolase gene by polymorphisnis associated with adult-type hypolactasia, Gut 52, 647-52 (2003); Olds, L. C. & Sibley, E., Lactase persistence DNA variant enhances lactase promoter activity in vitro: functional role as a cis regulatory element, Hum Mol Genet 12, 2333-40 (2003); Troelsen, J. T., Olsen, J., Moller, J. & Sjostrom, H., An upstream polymorphism associated with lactase persistence has increased enhancer activity, Gastroenterology 125, 1686-94 (2003). Lewinsky, R. H. et al., T-13910 DNA variant associated with lactase persistence interacts with Oct-1 and stimulates lactase promoter activity in vitro, Hum Mol Genet 14, 3945-53 (2005).

It has been hypothesized that natural selection has played a major role in determining the frequencies of LP in different human populations since the development of cattle domestication in the Middle East and North Africa ˜7.5-9 kya. See Hollox, E. & Swallow, D. M., The Genetic Basis of Common Diseases (eds. King, R. A., Rotter, J. I. & Motulsky, A. G.) 250-265 (Oxford University Press, Oxford, 2002); Durham, W. H. Coevolution: Genes, Culture, and Human Diversity (Stanford University Press, Stanford, 1992); Poulter, M. et al., The causal element for the lactase persistence/non-persistence polymorphism is located in a 1 Mb region of linkage disequilibrium in Europeans, Ann Hum Genet 67, 298-311 (2003); Hollox, E. J. et al., Lactase haplotype diversity in the Old World., Am J Hum Genet 68, 160-172 (2001); Bersaglieri, T. et al., Genetic signatures of strong recent positive selection at the lactase gene, Am J Hum Genet 74, 1111-20 (2004); Myles, S. et al., Genetic evidence in support of a shared Eurasian-North African dairying origin, Hum Genet 117, 34-42 (2005); The International HapMap Consortium, A haplotype map of the human genome, Nature 437, 1299-1320 (2005), Voight, B. F., Kudaravalli, S., Wen, X. & Pritchard, J. K, A map of recent positive selection in the human genome, PLoS Biol 4, e72 (2006); Nielsen, R. et al., A scan for positively selected genes in the genomes of humans and chimpanzees, PLoS Biol 3, e170 (2005). A region of extensive LD spanning >1 Mbp has been observed on European chromosomes with the T-13910 mutation, consistent with recent positive selection. See Poulter, M. et al., The causal element for the lactase persistence/non-persistence polymorphism is located in a 1 Mb region of linkage disequilibrium in Europeans, Ann Hum Genet 67, 298-311 (2003); Bersaglieri, T et al., Genetic signatures of strong recent positive selection at the lactase gene, Am J Hum Genet 74, 1111-20 (2004); The International HapMap Consortium, A haplotype map of the human genome, Nature 437, 1299-1320 (2005), Voight, B. F., Kudaravalli, S., Wen, X. & Pritchard, J. K, A map of recent positive selection in the human genome, PLoS Biol 4, e72 (2006); Nielsen, R. et al., A scan for positively selected genes in the genomes of humans and chimpanzees, PLoS Biol 3, e170 (2005). Based on the breakdown of LD on chromosomes with the T-13910 mutation, Bersaglieri et al. estimate that this mutation arose within the past ˜2,000-20,000 years within Europeans, likely in response to strong selection for the ability to digest milk as adults. See Bersaglieri, T. et al., Genetic signatures of strong recent positive selection at the lactase gene, Am J Hum Genet 74, 1111-20 (2004).

Although the T-13910 variant is the likely causal mutation of the LP trait in Europeans, analyses of this SNP in ethnically and geographically diverse African populations indicated that it is present (and at low frequency <14%) in only a few West African pastoralist populations, such as the Fulani (or Fulbe) and Hausa from Cameroon. See Myles, S. et al., Genetic evidence in support of a shared Eurasian-North African dairying origin, Hum Genet 117, 34-42 (2005); Mulcare, C. A. et al., The T allele of a single-nucleotide polymorphism 13.9 kb upstream of the lactase gene (LCT) (C-13.9 kbT) does not predict or cause the lactase-persistence phenotype in Africans, Am J Hum Genet 74, 1102-10 (2004); Coelho, M. et al., Microsatellite variation and evolution of human lactase persistence, Hum Genet 117, 329-39 (2005). It is absent in all other African populations tested, including East African pastoralist populations with a high prevalence of the LP trait. Mulcare, C. A. et al., The T allele of a single-nucleotide polymorphism 13.9 kb upstream of the lactase gene (LCT) (C-13.9 kbT) does not predict or cause the lactase-persistence phenotype in Africans, Am J Hum Genet 74, 1102-10 (2004). Thus, it is believed that the LP trait has evolved independently in most African populations, due to a distinct genetic mutation. See Myles, S. et al., Genetic evidence in support of a shared Eurasian-North African dairying origin, Hum Genet 117, 34-42 (2005); Mulcare, C. A. et al., The T allele of a single-nucleotide polymorphism 13.9 kb upstream of the lactase gene (LCT) (C-13.9 kbT) does not predict or cause the lactase-persistence phenotype in Africans, Am J Hum Genet 74, 1102-10 (2004); Coelho, M et al., Microsatellite variation and evolution of human lactase persistence, Hum Genet 117, 329-39 (2005).

Yet, the problematic condition referred to as adult-type hypolactasia or lactase non-persistence continues to affect most populations including Africans. As a result, lactose intolerance is a frequent phenomenon resulting in a potentially severe digestive disorder from milk and dairy products in the afflicted individuals. Not only do individuals afflicted with lactase non-persistence experience an inability to enjoy dairy products such as milk, but lactase non-persistence is a major cause of non-specific abdominal symptoms (e.g., stomach pain). Moreover, lactose intolerance is commonly considered a disease which can be treated only symptomatically. However, since lactose intolerance results from a deficiency in LPH (the lactase enzyme), there is the possibility of administering LPH to afflicted individuals so as to compensate for the lactase deficiency. This is commonly done by the individual ingesting LPH in the form of capsules, tablets or solution.

Accordingly, there are methods for diagnosing lactose intolerance. One of the most common is the H₂ breath test. With this diagnostic, an individual drinks a solution of 50 g lactose in water. The hydrogen subsequently exhaled by the individual is measured repeatedly by gas chromatography over 4 hours. In addition to the H₂ breath test, there is also a lactose tolerance test in which 50 g of lactose in water is administered to subjects on empty stomachs, followed by measuring the blood glucose level in such subjects over several hours. Unless the lactose is completely cleaved enzymatically, the glucose level remains low and thus confirms the lactose intolerance. However, the known diagnostic methods have several disadvantages. One disadvantage is the relatively large amount of lactose that must be delivered to the afflicted individuals, which may lead to more discomfort and pain from those individuals suffering from lactose intolerance. The assaying of the blood glucose levels is disadvantageous in that the blood glucose level may be changed by secondary factors, such as increased release of adrenalin due to stress. Moreover, the diagnostic methods known in the art require the sampling and measurement of several samples over an extended period of time, which is inconvenient, stressful and costly for the tested individual. Furthermore, while it has been hypothesized that it may be possible to test for lactose intolerance with ¹³C-labeled lactose, tests such as the lactose breath test would require a large amount of 13C-labeled lactose, which is exceedingly expensive.

In sum, the state of the art provides no biochemical test which is accurate, quick, cost-effective and convenient for tested individuals. Investigations into the cause of lactase persistence and non-persistence at the genomic level have also been unsuccessful. For example, to date, the sequencing of the coding and promoter regions of the LPH gene has revealed no DNA-variations which correlate with lactase persistence and non-persistence.

Therefore, a need in the art for an accurate, cost-effective and convenient test remains. This need is especially important for those populations in which the causative factor(s) for lactase persistence/non-persistence has yet to be investigated and thus are hardly understood. Accordingly, as described herein, new genotype/phenotype associations have been investigated. These investigations have revealed novel mutations associated with the LP trait that arose independently from the European T-13910 mutation and result in enhanced transcription activity in LCT promoter-driven reporter gene assays. In view of these investigations as described herein in detail, the problems as well as the needs in the art may now be addressed.

### SUMMARY OF THE INVENTION

According to an exemplary embodiment, the present invention generally relates to a method for determining an individual's predisposition for lactase non-persistence, said method comprising: determining the absence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene; wherein the absence of the one or more single nucleotide polymorphisms indicates that the individual has a predisposition for lactase non-persistence.

According to another exemplary embodiment, the present invention generally relates to a method for determining an individual's predisposition for lactase persistence, said method comprising: determining the presence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene; wherein the presence of the one or more single nucleotide polymorphisms indicates that the individual has a predisposition for lactase persistence.

According to another exemplary embodiment, the present invention generally relates to a method for genotyping an individual comprising determining the absence or presence of a variant allele containing a single nucleotide polymorphism within a gene associated with the expression of lactase-phlorizin hydrolase, in a biological sample from the individual, wherein the single nucleotide polymorphism is selected from the group consisting essentially of C-14010, G-13915 and G-13907, measured from the start of the LCT gene.

According to another exemplary embodiment, the present invention generally relates to an isolated nucleic acid molecule comprising a variant MCM 6 nucleotide sequence, a variant nucleotide sequence comprising intron 13 of MCM 6, or a sequence complementary thereto, wherein the said variant nucleotide sequence comprises at least a fragment of SEQ ID NO 1, wherein at least one of the following conditions applies: a) the nucleotide at position 13907 is guanine; b) the nucleotide at position 13915 is guanine; or c) the nucleotide at position 14010 is cytosine.

### BRIEF DESCRIPTION OF THE DRAWINGS

Examples of the invention are illustrated, by way of example and without limitation, in the accompanying figures, wherein:

FIG. 1 shows a map of the LCT and MCM 6 gene region and location of genotyped single nucleotide polymorphisms, in accordance with an exemplary embodiment of the present invention.

FIG. 2 shows a map of phenotype and genotype proportions for several population groups, in accordance with an exemplary embodiment of the present invention.

FIG. 3 shows genotype/phenotype association for G/C-14010, T/G-13915 and C/G-13907, in accordance with an exemplary embodiment of the present invention.

FIG. 4 shows haplotype networks consisting of 55 single nucleotide polymorphisms spanning a 98 kb region encompassing LCT and MCM 6, in accordance with an exemplary embodiment of the present invention.

FIG. 5 shows a luciferase assay of LCT promoter and MCM6 introns, in accordance with an exemplary embodiment of the present invention.

FIG. 6 shows a comparison of tracts of homozygous genotypes flanking the lactase persistence associated single nucleotide polymorphisms, in accordance with an exemplary embodiment of the present invention.

FIG. 7 shows plots of the extent and decay of haplotype homozygosity in the region surrounding the C-14010 allele, in accordance with an exemplary embodiment of the present invention.

FIG. 8 shows the distribution of phenotype values for a pooled African dataset, in accordance with an exemplary embodiment of the present invention.

FIG. 9 shows linear regression based tests of association for each polymorphic single nucleotide polymorphism over a pooled dataset, in accordance with an exemplary embodiment of the present invention.

FIG. 10 shows an estimation of the degree of dominance for G/C-14010, T/G-13915 and C/G-13907, in accordance with an exemplary embodiment of the present invention.

FIG. 11 shows plots of the extent and decay of haplotype homozygosity in the region surrounding the G-r2322813, G-13907 and G-13915 alleles, in accordance with an exemplary embodiment of the present invention.

### DETAILED DESCRIPTION OF THE PREFERRED EMBODIMENTS

For simplicity and illustrative purposes, the present invention may be discussed by way of examples of the methods, tests, kits and/or nucleic acid molecules described. In the following description, numerous specific details and examples are set forth in order to provide a thorough understanding of the present invention. It will be apparent however, to one of ordinary skill in the art, that the present invention may be practiced without limitation to these specific details and examples. In other instances, well known aspects are not described in detail so as not to unnecessarily obscure the understanding of the present invention.

In accordance with an exemplary embodiment, the present invention generally relates to methods for determining a predisposition (e.g., genetic predisposition) for lactase non-persistence. For example, in one preferred embodiment the present invention generally relates to methods for determining a predisposition for lactase non-persistence based on determining or identifying the absence of a variant allele associated with the expression of the enzyme lactase phlorizin hydrolase in an individual, as opposed to the presence of a normal, or “wild type,” allele for lactase phlorizin hydrolase. In another preferred embodiment the present invention generally relates to methods for determining a predisposition for lactase persistence based on determining or identifying the presence of a variant allele associated with the expression of the enzyme lactase phlorizin hydrolase in an individual, as opposed to the absence of a normal, or “wild type,” allele for lactase phlorizin hydrolase. The variant allele may differ from the “wild type” allele by a single nucleotide polymorphism, or SNP, at one or more points in the nucleotide sequence. Those of ordinary skill in the art will recognize that a SNP is a DNA sequence (nucleotide sequence) variation occurring when a single nucleotide in the “wild type” allele is replaced or substituted by a different nucleotide in the variant allele. For example, cytosine may be replaced by guanine. A SNP may occur at only one point in the allele, or multiple noncontiguous sites in the allele. A SNP variant allele that is common in one geographical or ethnic group may be much rarer in another.

Single nucleotide polymorphisms can be identified by sequencing a DNA strand (nucleotide sequence) from an individual and comparing the sequenced DNA strand (nucleotide sequence) to a known “wild type” version of the same allele. Those of ordinary skill in the art will recognize that a sample of DNA may be obtained via polymerase chain reaction (PCR). PCR is used to amplify specific regions of a DNA strand (nucleotide sequence). In one example, PCR, as typically practiced, involves a DNA template that contains the region of the DNA fragment to be amplified and one or more primers, which are complementary to the 5′ (five prime) and 3′ (three prime) ends of the DNA region that is to be amplified. A DNA polymerase and a mixture of deoxynucleotide triphosphates are used to synthesize new DNA molecules which match the sequence of the DNA template. The resulting amplified DNA may then be sequenced by methods which are well known in the art, and compared to the “wild type” DNA sequence, so as to identify polymorphisms.

Lactose intolerance (or hypolactasia) is the term used to describe a decline in the ability to digest lactase, an enzyme needed for proper metabolization of lactose (a sugar that is a constituent of milk and other dairy products), in human beings. The inability to digest lactose is typically diagnosed in several ways. For example, the Lactose Tolerance Test (LTT) measures rise in blood glucose levels following consumption of 50 g of lactose (equivalent to ˜1-2 liters of cow's milk). Individuals exhibiting the “Lactase Persistent” phenotype exhibit a rise of >1.7 mM/L in blood glucose level, while individuals exhibiting the “Lactase Non-Persistent” phenotype exhibit a rise of <1.1 mM/L in blood glucose level. Alternatively, lactase enzyme activity may be measured directly by intestinal biopsy, or the concentration of urinary galactose after administration of lactose. Lactase non-persistence is a type of lactose intolerance that normally develops after weaning in cultures where adult consumption of milk is rare (often referred to as primary lactose intolerance); these phenotypic tests are normally unable to distinguish between lactase non-persistence and lactose intolerance arising from other causes, such as gastrointestinal disease or parasitic infection (secondary lactose intolerance); or an inability to express enzymes needed for lactose digestion at birth. In general, as exemplified in the various embodiments discussed herein, identification of a variant allele having a specified single point polymorphism can be associated with a genetic predisposition toward lactase persistence. Conversely, the lack of such a variant allele in a lactose intolerant individual may indicate that lactose intolerance arises from other genetic causes or from infection. Accordingly, in accordance with exemplary embodiments as described herein, the present invention generally relates to methods for determining a predisposition (e.g., genetic predisposition) for lactase intolerance, by determining an individual's predisposition (e.g., genetic predisposition) for lactase non-persistence.

In accordance with another exemplary embodiment, the present invention generally relates to methods for determining a predisposition (e.g., genetic predisposition) for lactase non-persistence, preferably for determining an individual's predisposition for lactose intolerance.

In accordance with a preferred exemplary embodiment, the present invention relates to a method for determining an individual's predisposition for lactase non-persistence, the method comprising: determining the absence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene; wherein the absence of the one or more single nucleotide polymorphisms indicates that the individual has a predisposition for lactase non-persistence. As used herein, the phrase “determining the absence” (e.g., of an allele or SNP) may be understood to include “determining the presence” (e.g., of that same allele or SNP).

For example, in one preferred embodiment, the method disclosed herein relates to a method for determining an individual's predisposition for lactase non-persistence. This method may be performed by determining the absence of at least one variant allele which differs from the “wild type” allele by the presence of at least one single nucleotide polymorphism within a gene associated with expression of lactase-phlorizin hydrolase. The gene is preferably the MCM 6 gene which is associated with the expression of lactase-phlorizin hydrolase, wherein the SNP is selected from the group consisting essentially of a cytosine nucleotide at position 14010 (C-14010), a guanine nucleotide at position 13915 (G-13915), a guanine nucleotide at position 13907 (G-13907) and combinations thereof, as measured upstream from the start of the LCT gene. In one example, the absence of one or more of these SNPs indicates that the individual has a predisposition for lactase non-persistence. In another example, the absence of each of these SNPs indicates that the individual has a predisposition for lactase non-persistence. Those of ordinary skill in the art will recognize that in view of the investigations of the present invention, wherein it has now been determined that the presence of the variant allele having one or more the SNPs C-14010, G-13915 and G-13907 indicates a predisposition for lactase persistence (as discussed herein), an individual may also be tested for lactase non-persistence by determining the presence of a “wild type” allele of a gene associated with expression of lactase-phlorizin hydrolase. The “wild type” allele of the gene associated with the expression of lactase-phlorizin hydrolase may be characterized by the presence of a guanine nucleotide at position 14010 (G-14010), a thymine nucleotide at position 13915 (T-13915) and a cytosine nucleotide at position 13907 (C-13907), as measured upstream from the start of the LCT gene.

In view of the above, those of ordinary skill in the art will also recognize that the presence of the variant allele comprising one or more SNP, wherein the SNP is selected from the group consisting essentially of C-14010, G-13915 and G-13907, may indicate that the individual has a predisposition for lactase persistence. One or more SNP selected from the group consisting essentially of C-14010, G-13915 and G-13907 may indicate that the individual has a predisposition for lactase persistence as compared to the absence of one or more SNP (e.g., all of the SNPs) or the presence of the wild type allele of the gene associated with the expression of lactase-phlorizin hydrolase which may be characterized by the presence of G-14010, T-13915 and C-13907, as measured upstream from the start of the LCT gene. That is, the presence of an allele of the gene associated with the expression of lactase-phlorizin hydrolase having at least one of C-14010, G-13915 and G-13907, as measured from the start of the LCT gene, may indicate that the individual has a predisposition for lactase persistence as compared to the absence the allele having one or more of G-14010, T-13915 and C-13907.

Those of ordinary skill in the art will recognize that in accordance with an exemplary embodiment as described herein, the variant allele comprises one or more SNPs, wherein the SNPs are selected from the group consisting essentially of C-14010, G-13915 and G-13907, measured from the start of the LCT gene. In accordance with another exemplary embodiment as described herein, the variant allele comprises a single SNP, wherein the SNP is a cytosine nucleotide at position 14010 (C-14010), measured upstream from the start of the LCT gene. In accordance with another exemplary embodiment as described herein, the variant allele comprises a single SNP, wherein the SNP is a guanine nucleotide at position 13915 (G-13915), measured upstream from the start of the LCT gene. In accordance with another exemplary embodiment as described herein, the variant allele comprises a single SNP, wherein the SNP is a guanine nucleotide at position 13907 (G-13907), measured upstream from the start of the LCT gene. Preferably, the SNP is C-14010, as measured upstream from the start of the LCT gene.

In accordance with exemplary embodiments described herein, a predisposition for lactase non-persistance may be determined by determining the absence of a variant allele having one or more SNP C-14010, G-13915 and G-13907, by amplifying a nucleotide sequence of the gene associated with the expression of lactase-phlorizin hydrolase; and detecting the absence of the SNP in the amplified nucleotide sequence. Alternatively, a predisposition for lactase non-persistance may be determined by determining the presence of a “wild type” allele of the MCM 6 gene which lacks SNPs at each of positions 14010, 13915, and 13907, by amplifying a nucleotide sequence of the MCM6 gene associated with the expression of lactase-phlorizin hydrolase; and detecting the presence of the “wild type” MCM 6 gene as described herein. Furthermore, in accordance with the exemplary embodiments described herein, the presence or absence of the variant allele containing one or more SNP may be detected by sequencing the amplified nucleotide sequence. The sequencing of the amplified nucleotide sequence is described in further detail herein.

In accordance with the exemplary embodiments described herein, the gene associated with the expression of lactase-phlorizin hydrolase is MCM 6.

In accordance with the exemplary embodiments described herein, those of ordinary skill in the art will recognize that the methods of the present invention may also be used as a method of test for determining lactose intolerance or tolerance. For example, those of ordinary skill in the art will recognize that, in accordance with one preferred embodiment, the methods described herein for determining a predisposition for lactase non-persistence/persistence may also be used to determine a predisposition for lactose intolerance/tolerance.

In accordance with another exemplary embodiment, the present invention generally relates to methods for determining a predisposition (e.g., genetic predisposition) for lactase persistence, preferably for determining an individual's predisposition for lactose tolerance.

In accordance with a preferred exemplary embodiment, the present invention relates to a method for determining an individual's predisposition for lactase persistence, said method comprising: determining the presence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene; wherein the presence of the one or more single nucleotide polymorphisms indicates that the individual has a predisposition for lactase persistence. As used herein, the phrase “determining the presence” (e.g., of an allele or SNP) may be understood to include “determining the absence” (e.g., of that same allele or SNP).

For example, in one preferred embodiment, the method disclosed herein relates to a method for determining an individual's predisposition for lactase persistence. Such a method may be described wherein an individual may be tested for lactase persistence by determining the presence of at least one variant allele which differs from the “wild type” allele by the presence of at least one single nucleotide polymorphism within a gene associated with expression of lactase-phlorizin hydrolase. The single nucleotide polymorphism is preferably selected from the group consisting essentially of a cytosine nucleotide at position 14010 (C-14010), a guanine nucleotide at position 13915 (G-13915), a guanine nucleotide at position 13907 (G-13907) and combinations thereof, as measured upstream from the start of the LCT gene. As discussed herein, the presence of one or more of these single nucleotide polymorphisms may indicate that the individual has a predisposition for lactase persistence. Preferably, the gene associated with the expression of lactase-phlorizin hydrolase is MCM 6.

In one example of the present invention, the method for determining an individual's predisposition for lactase comprises determining the presence of the single nucleotide polymorphism C-14010. As discussed herein, the presence of the SNP substituting cytosine for “wild type” guanine at position 14010, as measured relative to the start of the LCT gene (G/C-14010), may indicate lactase persistence in tested populations. In another example, the method for determining an individual's predisposition for lactase comprises determining the presence of the single nucleotide polymorphism G-13915. As discussed herein, the presence of a SNP substituting guanine for “wild type” thymine measured relative to the start of the LCT gene (T/G-13915), may indicate lactase persistence in tested populations. In another example, the method for determining an individual's predisposition for lactase comprises determining the presence of the single nucleotide polymorphism is G-13907. As discussed herein, the presence of a SNP substituting guanine for “wild type” cytosine at position 13907 measured relative to the start of the LCT gene (C/G-13907), may indicate lactase persistence in tested populations.

In another exemplary embodiment of the present invention, the absence of at least one of the one ormore single nucleotide polymorphisms, C-14010, G-13915 and G-13907, indicates that the individual has a predisposition for lactase non-persistence. In one example, the presence of an allele of the gene associated with the expression of lactase-phlorizin hydrolase having at least one single nucleotide polymorphism selected from G-14010, T-13915 and C-13907, as measured from the start of the LCT gene, indicates that the individual has a predisposition for lactase non-persistence as compared to the presence of one or more of the variant alleles having the single nucleotide polymorphism. In another example, the presence of each of the single nucleotide polymorphisms G-14010, T-13915 and C-13907, as measured from the start of the LCT gene, indicates that the individual has a predisposition for lactase non-persistence as compared to the presence of one or more of the variant alleles having the single nucleotide polymorphism.

In accordance with the exemplary embodiments described herein, those of ordinary skill in the art will recognize that the methods of the present invention may also be used as a method of test for determining lactose intolerance or tolerance. For example, those of ordinary skill in the art will recognize that, in accordance with one preferred embodiment, the methods described herein for determining a predisposition for lactase non-persistence/persistence may also be used to determine a predisposition for lactose intolerance/tolerance.

In accordance with another exemplary embodiment, the methods of the present invention comprise determining the presence of the single nucleotide polymorphism by amplifying a nucleotide sequence comprising the variant allele having at least one single nucleotide polymorphism selected from the group consisting essentially of C-14010, G-13915 and G-13907; and detecting the presence of the single nucleotide polymorphism in the amplified nucleotide sequence. Amplification is preferably carried out via polymerase chain reaction, selectively amplifying a specific region(s) of DNA (nucleotide sequence), preferably the variant allele, preferably the variant allele of the MCM 6 gene. In other words, PCR may be used to isolate desired sections of DNA (nucleotide sequence) from whole genomic material. The amplified DNA (nucleotide sequence) may then be used to detect the presence of a single nucleotide polymorphism at position 14010, at position 13915, and/or at position 13907, as measured upstream from the start of the LCT gene. In one example, if the amplified DNA corresponds to the “wild type” version of intron 13 of the MCM 6 gene, i.e., the amplified DNA has a guanine nucleotide at position 14010 (G-14010), measured upstream from the start of the LCT gene, a thymine nucleotide at position 13915 (T-13915) and a cytosine nucleotide at position 13907 (C-13907), the individual may be diagnosed as having a predisposition toward lactase non-persistence. In another example, if the amplified DNA corresponds to a variant version of intron 13 of the MCM 6 gene, i.e., if the amplified DNA has a at least one of a cytosine nucleotide at position 14010 (G-14010), measured upstream from the start of the LCT gene, a guanine nucleotide at position 13915 (T-13915) and a guanine nucleotide at position 13907 (C-13907), the individual may be diagnosed as having a genetic predisposition toward lactase persistence.

In accordance with exemplary embodiments, the present invention comprises sequencing the amplified nucleotide sequence. Preferably, detecting the presence of a single nucleotide polymorphism in the amplified nucleotide sequence includes sequencing the amplified nucleotide sequence. Those of ordinary skill in the art will recognize that amplified DNA (nucleotide sequence) prepared by PCR may be used for DNA sequencing, as well as the detection of a predisposition for genetic disease. Those of ordinary skill in the art will further recognize that DNA sequencing may be carried out by any of various methods which are well known in the art. Accordingly, once a DNA sequence is known, the presence of a single nucleotide polymorphism associated with lactase persistence may be determined by identifying the presence of at least one of a cytosine base at position 14010 measured relative to the start of the LCT gene, a guanine base at position 13915 measured relative to the start of the LCT gene, and/or a guanine base at position 13907 measured relative to the start of the LCT gene.

In accordance with the exemplary embodiments described herein, the gene associated with the expression of lactase-phlorizin hydrolase is MCM 6.

As discussed above, exemplary embodiments of the present invention include determining a predisposition for lactase persistence (lactose tolerance) and/or lactase non-persistence (lactose intolerance). For instance, in one preferred example of the present invention, a DNA strand (nucleotide sequence) containing the MCM 6 gene, intron 13 of the MCM 6 gene or a sequence comprising a base pair sequence including position 14010, measured relative to the start of the LCT gene; position 13915, measured relative to the start of the LCT gene, and/or position 13907, measured relative to the start of the LCT gene, is obtained from an individual. Preferably, the individual is suspected of having a predisposition for lactose intolerance. The DNA strand (nucleotide sequence) is amplified and the sequence determined. Once the DNA sequence is known, the presence or absence of a single nucleotide polymorphism associated with lactase persistence may be determined by identifying the presence of each of a guanine base at position 14010 measured relative to the start of the LCT gene, a thymine base at position 13915 measured relative to the start of the LCT gene, and a cytosine base at position 13907 measured relative to the start of the LCT gene. Upon determining the presence of one or more single nucleotide polymorphism, an individual having a predisposition for lactase persistence (lactose tolerance) may be identified. Upon determining the absence one or more single nucleotide polymorphism, an individual having a predisposition for lactase non-persistence (lactose intolerance) may be identified.

In accordance with another exemplary embodiment, the present invention generally relates to methods for determining a predisposition (e.g., genetic predisposition) for lactase persistence and/or non-persistence. For example, the determination of an individual's predisposition for lactase non-persistence or non-persistence may be performed by collecting a DNA strand (nucleotide sequence) comprising the MCM 6 gene, intron 13 of the MCM 6 gene or a base pair sequence which includes the positions 14010, 13915 and/or 13907, measured relative to the start of the LCT gene. Determination of the DNA sequence may be performed by sequencing the DNA strand. Furthermore, as will be recognized by those of ordinary skill in the art, determination of the genotype may be determined by conducting a hybridization assay, by hybridizing the sample DNA strand to a DNA probe having a known sequence. In view of the discussion herein, those of ordinary skill in the art will recognize that useful DNA probes for such hybridization assay may include, but are not limited to, one or more of the following:

A) A DNA probe complementary to “wild type” intron 13 of the MCM 6 gene;

B) A DNA probe complementary to a variant form of intron 13 of the MCM 6 gene having a cytosine at position 14010;

C) A DNA probe complementary to a variant form of intron 13 of the MCM 6 gene having a guanine at position 13915; and

D) A DNA probe complementary to a variant form of intron 13 of the MCM 6 gene having a guanine at position 13907.

In accordance with the exemplary embodiments described herein, preferential hybridization to probe A indicates that the individual from whom the sample DNA came has a genetic predisposition toward lactase non-persistence. Also in accordance with the exemplary embodiments described herein, preferential hybridization to one or more of probes B, C, and D indicates that the individual from whom the sample DNA came has a genetic predisposition toward lactase persistence.

In accordance with another exemplary embodiment, the present invention generally relates to methods for genotyping an individual. In accordance with another exemplary embodiment, the present invention comprises a method for genotyping an individual, the method comprising determining the absence or presence of a single nucleotide polymorphism within a gene associated with expression of lactase-phlorizin hydrolase.

For example, in accordance with one preferred embodiment, the present invention relates to methods for genotyping an individual comprising determining the absence or presence of a variant allele containing a single nucleotide polymorphism within a gene associated with the expression of lactase-phlorizin hydrolase, in a biological sample from the individual, wherein the single nucleotide polymorphism is selected from the group consisting essentially of C-14010, G-13915 and G-13907, measured from the start of the LCT gene. In one example, the absence of one or more of the single nucleotide polymorphisms C-14010, G-13915 and G-13907 indicates that the individual has a predisposition for lactase non-persistence as compared to the presence of one or more of the single nucleotide polymorphisms. In another example, the presence of one or more of the single nucleotide polymorphisms C-14010, G-13915 and G-13907 indicates that the individual has a predisposition for lactase persistence as compared to the absence of one or more of the single nucleotide polymorphisms.

In one preferred embodiment, the determination of an individual genotype (e.g., for determining the absence or presence of a single point polymorphism associated with lactase persistence) may be performed by collecting a DNA strand (nucleotide sequence) from the individual. The DNA strand (nucleotide sequence) preferably comprises the MCM 6 gene, intron 13 of the MCM 6 gene or a base pair sequence which includes the positions 14010, 13915, and 13907, measured relative to the start of the LCT gene. Determination of the genotype may be performed by amplifying and sequencing the DNA strand (nucleotide sequence). For example, the absence or presence of the single nucleotide polymorphism may be determined by amplifying a nucleotide sequence of the gene associated with the expression of lactase-phlorizin hydrolase; and detecting the absence or presence of the single nucleotide polymorphism in the amplified nucleotide sequence, wherein the step detecting comprises sequencing the amplified nucleotide sequence. Once sequenced, the DNA strand (nucleotide sequence) may be reviewed for the presence of variant alleles having single point polymorphisms at positions 14010, 13915, and 13907. In one example, one or more of the polymorphisms C-14010, G-13915 and G-13907 is identified, thereby identifying the individual as having a genotype corresponding to a predisposition for lactase persistence. In another example, the polymorphisms C-14010, G-13915 and G-13907 are absent, thereby identifying the individual as having a genotype corresponding to a predisposition for lactase non-persistence.

In another preferred embodiment, the determination of the genotype may be determined via hybridization assay, by hybridizing the sample DNA strand to a DNA probe having a known sequence. In view of the discussion herein, those of ordinary skill in the art will recognize that useful DNA probes include, but are not limited to, one or more of the following:

A) A DNA probe complementary to “wild type” intron 13 of the MCM 6 gene;

B) A DNA probe complementary to a variant form of intron 13 of the MCM 6 gene having a cytosine at position 14010;

C) A DNA probe complementary to a variant form of intron 13 of the MCM 6 gene having a guanine at position 13915; and

D) A DNA probe which is complementary to a variant form of intron 13 of the MCM 6 gene having a guanine at position 13907.

In one example, preferential hybridization to probe A indicates that the individual from whom the sample DNA came has a genotype corresponding to a genetic predisposition toward lactase non-persistence. In another example, preferential hybridization to one or more of probes B, C, and D indicates that the individual from whom the sample DNA came has a genotype corresponding to a genetic predisposition toward lactase persistence.

In accordance with an exemplary embodiment of the present invention, the absence of one or more of the single nucleotide polymorphisms, as determined by DNA sequencing or preferential hybridization to probe A above (or other appropriate probe), indicates that the individual has a predisposition for lactase non-persistence as compared to the presence of one or more of the single nucleotide polymorphisms.

In accordance with an exemplary embodiment of the present invention, the presence of one or more of the single nucleotide polymorphisms, as determined by DNA sequencing or preferential hybridization to at least one of probes B, C, or D above (or other appropriate probe), indicates that the individual has a predisposition for lactase persistence as compared to the absence of one or more of the single nucleotide polymorphisms.

In accordance with an exemplary embodiment of the present invention, the single nucleotide polymorphism is the presence of cytosine at position 14010 (C-14010).

In accordance with an exemplary embodiment, the methods of the present invention preferably comprise: determining the absence or presence of the single nucleotide polymorphism by amplifying a nucleotide sequence of the gene associated with encoding for lactase-phlorizin hydrolase; and detecting the absence or presence of the single nucleotide polymorphism in the amplified nucleic acids. In one example, detection of the absence or presence of the single nucleotide polymorphism may be done by sequencing the amplified nucleic acids. In another example, detection of the absence or presence of the single nucleotide polymorphism may be determined by a hybridization assay to one or more of probes A, B, C, and D above.

In accordance with another exemplary embodiment, the present invention generally relates to a nucleic acid molecule (e.g., an isolated nucleic acid molecule) comprising a variant MCM 6 nucleotide sequence. In accordance with another exemplary embodiment, the present invention generally relates to kits, tests and/or for determining a predisposition for lactase non-persistence, non-persistence and/or lactose intolerance. In accordance with another exemplary embodiment, the present invention generally relates to vectors and/or transfected host cells comprising the nucleic acid molecules in accordance with the present invention.

For example, in accordance with an exemplary embodiment, the present invention relates to an isolated nucleic acid molecule comprising an isolated nucleic acid molecule comprising a variant MCM 6 nucleotide sequence, a variant nucleotide sequence comprising intron 13 of MCM 6, or a sequence complementary thereto, wherein the said variant nucleotide sequence comprises at least a fragment of SEQ ID NO 1, wherein at least one of the following conditions applies: a) the nucleotide at position 13907 is guanine; b) the nucleotide at position 13915 is guanine; or c) the nucleotide at position 14010 is cytosine. In a preferred example, the variant nucleotide sequence comprises a fragment of SEQ ID NO 1, wherein the fragment encompasses a base pair region encompassing at least one of the nucleotide positions 13907, 13915 and 14010 of SEQ ID NO 1, as measured relative to the start of the LCT gene. In another preferred example, the variant nucleotide sequence comprises the 103 base pair region from position −13907 to −14010 (as shown in SEQ ID NO 1), as measured relative to the start of the LCT gene. In another preferred example, the variant nucleotide sequence comprises intron 13 of the MCM 6 gene. In another preferred example, the variant nucleotide sequence comprises a fragment of intron 13 of the MCM 6 gene, wherein the fragment encompasses a base pair region encompassing at least one of the nucleotide positions 13907, 13915 and 14010 of SEQ ID NO 1, as measured relative to the start of the LCT gene.

In accordance with another exemplary embodiment of the present invention, the isolated nucleic acid molecule is located within a vector. Those of ordinary skill in the art will recognize a vector as a small DNA vehicle that carries a foreign DNA fragment. Insertion of the isolated nucleic acid molecule into the vector is preferably carried out by treating the DNA vehicle and the foreign DNA with the same restriction enzyme, and then ligating the fragments together. Those of ordinary skill in the art will recognize that several types of cloning vectors may be used. Plasmids and bacteriophages (such as phage λ) are perhaps most commonly used for such a purpose. However, other types of cloning vectors include bacterial artificial chromosomes (BACs) and yeast artificial chromosomes (YACs).

In accordance with a preferred exemplary embodiment, the vector is located within a transfected host cell. Those of ordinary skill in the art will recognize that transfection may be carried out, among other methods, by mixing a cationic lipid with vector to produce liposomes, which fuse with the cell plasma membrane and deposit the vector containing the isolated nucleic acid molecule inside. For many applications of transfection, it is sufficient if the transfected gene in the gene is only transiently expressed. Since the DNA introduced in the transfection process is usually not inserted into the nuclear genome, the foreign DNA is lost at the later stage when the cells undergo mitosis. If it is desired that the transfected gene actually remains in the genome of the cell and its daughter cells, a stable transfection must occur. To accomplish a stable transfection, it is preferred that additional foreign genetic material encoding an advantageous protein or other gene product may be co-transfected with the isolated MCM 6 gene. Some of the transfected cells will incorporate the foreign genetic material into their genome. The advantageous protein may, for example, provide the transfected cell with resistance to a toxin. If the toxin is then added to the cell culture, only those few cells with the gene for toxin resistance will survive. After applying this selection pressure for some time, only the cells with a stable transfection including the isolated MCM 6 gene remain and can be cultivated further.

In accordance with an exemplary embodiment of the present invention, the isolated nucleic acid molecule may be included as part of a kit for determining an individual's predisposition for lactase non-persistence, non-persistence, lactose tolerance and/or lactose intolerance. As discussed herein, the exemplar embodiments of the present invention enable genetic testing for “wild type” MCM 6 and its various polymorphic variants as discussed herein. A correlation to lactase persistence or the lack thereof in people having polymorphisms deviating from the normal or “wild type” phenotype may be drawn. Accordingly, a preferred kit of the present invention may include primers for amplifying DNA (nucleotide sequence) from an individual suspected of exhibiting lactase persistence, lactase non-persistence, lactose tolerance and/or lactose intolerance. Primers may be used to amplify the individual's DNA. The kit may also includes at least one of a DNA strand corresponding to “wild type” MCM 6 and a DNA strand corresponding to at least one MCM 6 gene having a single point nucleotide polymorphism at as described herein at position 13907, 13915 or 14010. Such kit components may allow for comparison of the properties of the amplified DNA to the properties of “wild type” MCM 6 or MCM 6 having a single point nucleotide polymorphism as provided in the kit. The comparison may be made, preferably, by gel electrophoresis, Northern blotting or Southern blotting.

In accordance with another exemplary embodiment, a kit may include at least one of a DNA strand which is complementary to “wild type” MCM 6 and a DNA strand which is complementary to at least one MCM 6 gene having a single point nucleotide polymorphism at as described herein at position 13907, 13915 or 14010. These may be probes A, B, C, and D as defined above. The kit may also preferably include a plate having at least one well for each DNA strand included in the kit. The kit may preferably include primers for amplifying DNA from an individual suspected of exhibiting lactase persistence, lactase non-persistence, lactose tolerance and/or lactose intolerance. These primers may be used to amplify the individual's DNA. The complementary sequences included in the kit are each preferably bound to the bottom of one of the wells in the plate by any of various means known in the art. Samples of the amplified DNA may be added to each well, and the samples examined for hybridization between the amplified DNA and the complementary DNA sequences included in the kit. In one example, hybridization of amplified DNA to a DNA strand which is complementary to “wild type” MCM 6 indicates a genetic predisposition toward lactase non-persistence. In another example, hybridization of amplified DNA to a DNA strand which is complementary to at least one MCM 6 gene having a single point nucleotide polymorphism indicates a genetic predisposition toward lactase persistence.

In view of the above, those of ordinary skill in the art will recognize a number of preferred tests and/or kits (and preferred components of such kits) for determining a predisposition for lactase non-persistence, non-persistence, lactose tolerance and/or lactose intolerance.

By way of example, without limitation, exemplary embodiments of the present invention may also be illustrated with reference to the drawings herein.

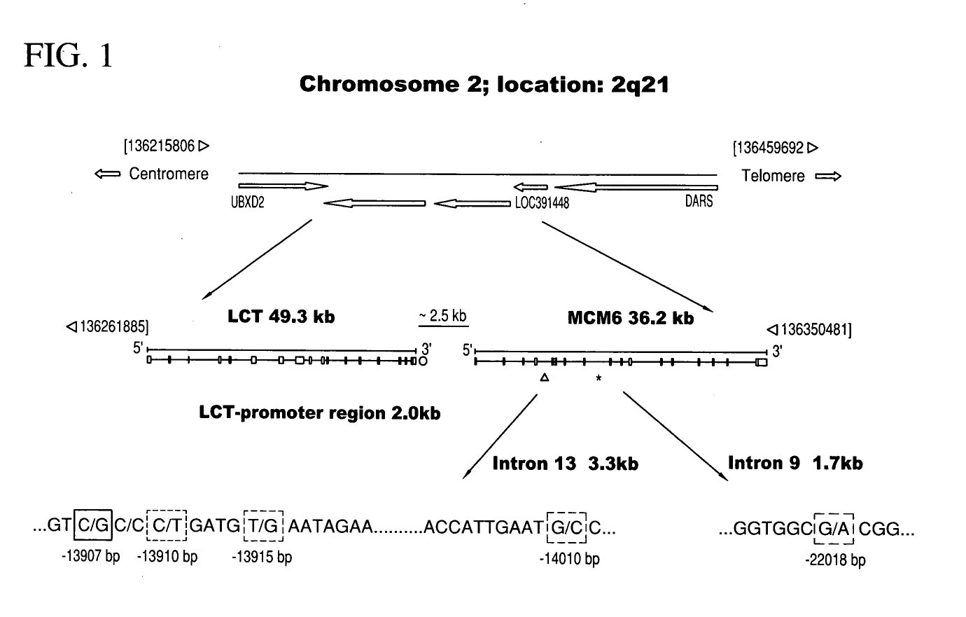

FIG. 1 shows a map of LCT and MCM 6 gene region and location of genotyped SNPs. In FIG. 1, (a) shows the distribution of 123 SNPs included in genotype analysis, (b) shows a map of the LCT and MCM 6 gene region, (c) shows a map of the MCM 6 gene, and (d) shows the location of LP-associated SNPs within introns 9 and 13 of the MCM 6 gene in African and European populations.

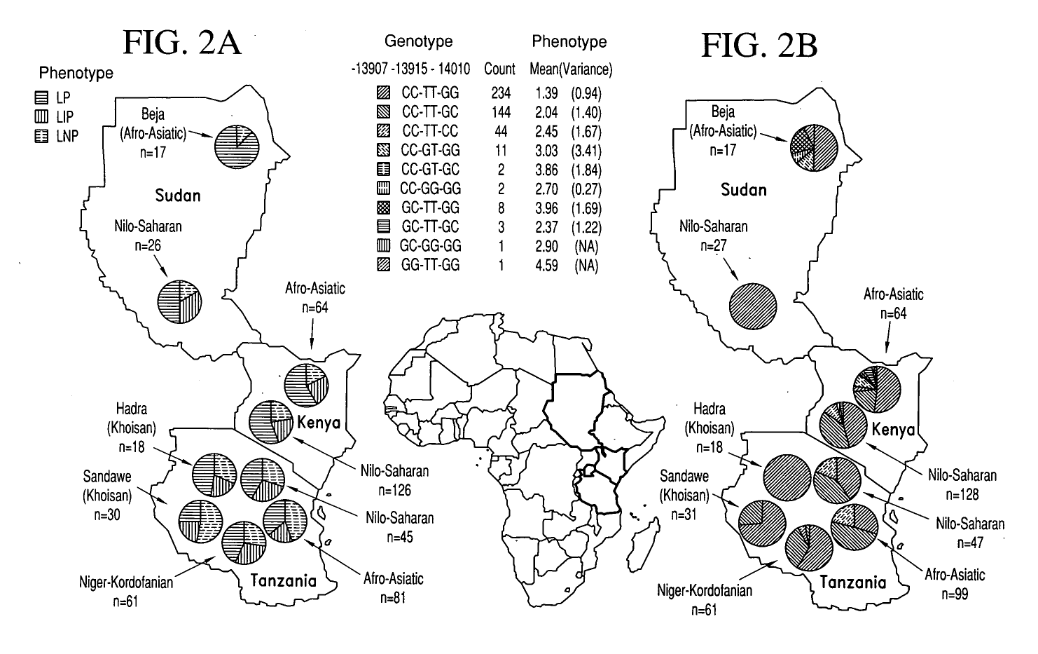

FIG. 2 shows a map of phenotype and genotype proportions for each population group considered in this study. In FIG. 2, A) shows pie charts representing the proportion of each phenotype by geographic region. LP indicates “Lactase Persistence”, LIP indicates “Lactase Intermediate Persistence”, LNP indicates “Lactase Non-Persistence”. Phenotypes were binned using an LTT test as follows: LP>1.7 mMol/L rise in blood glucose following digestion of 50 g lactose, 1.7 mMol/L>LIP>1.1 mMol/L, LNP<1.1 mMol/L. In FIG. 2, B) shows pie charts representing the proportion of compound genotypes for G/C-13907, T/G-13915, and C/G-14010 in each region. The pie charts are in the approximate geographic location of the sampled individuals.

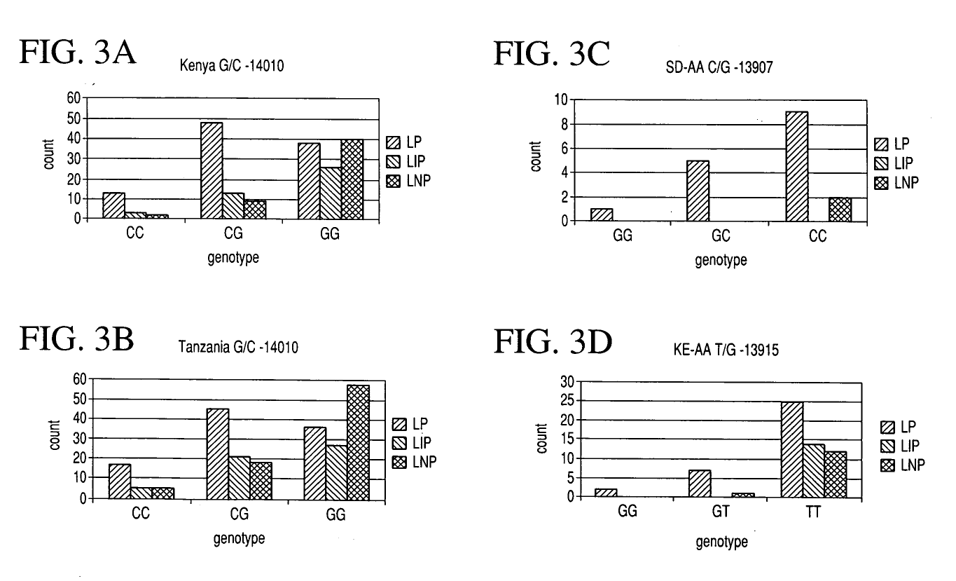

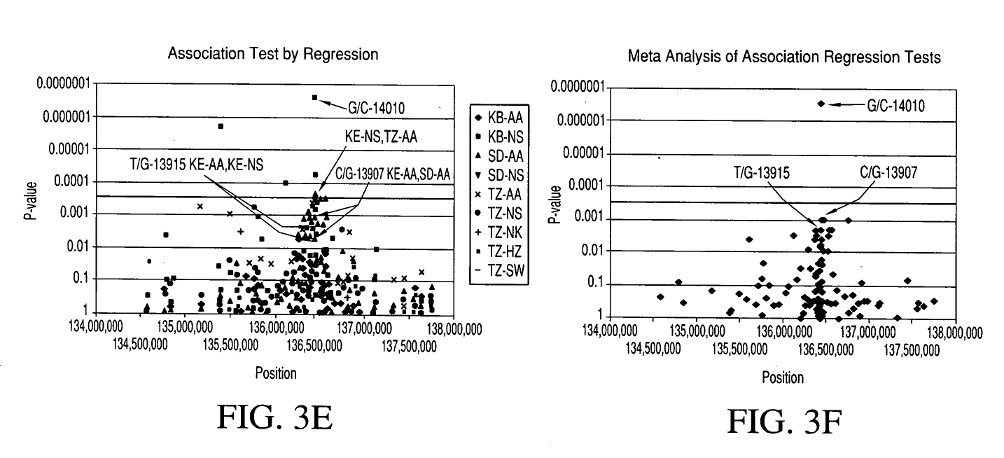

FIG. 3 shows the genotype/phenotype association for G/C-14010, T/G-13915, and C/G-13907. In FIG. 3, (a-d) shows the counts of the number of individuals in various genotype and phenotype classes in major geographic regions and/or populations in which they are most prevalent. Genotypes of G/C-14010 are plotted for all the Kenyan (a) and Tanzanian (b) individuals. Genotypes of C/G-13907 are plotted for the Sudanese Afro-Asiatic (c, SD-AA) and T/G-13915 for the Kenyan Afro-Asiatic (d, KE-AA) populations. A significant association is observed for the G/C-14010 SNP in Kenya (n=190, d.f.=4, Ω²=21.77, p=0.0002) and in Tanzania (n=231, d.f.=4, Ω²=21.90, p=0.0002). The association was less significant for the C/G-13907 SNP in the N. Sudanese (n=17, d.f.=2, chi-square 2.54; p=0.2808) and for the T/G-13915 SNP in Northern Kenyans (n=61, d.f.=4, Ω²=6.14, P=0.1889). It is noted that a large proportion of individuals who are homozygous for the ancestral G-14010, T-13915, and C-13907 alleles are classified as LP, indicating that there may be additional unidentified mutations associated with LP in these populations. (e) shows p-values from a linear regression based test of association for each polymorphic SNP genotyped in this study in each of the subpopulations. The dark line denotes the significance level after a conservative Bonferroni correction for the total number of SNPs tested. G/C-14010 is the most significant of all 123 genotyped SNPs in the Kenyan Nilo-Saharan (KE-NS) and Tanzanian Afro-Asiatic (TZ-AA) samples. C/G-13907 shows the strongest association (although not significant) compared to all other genotyped SNPs, in the Kenyan Afro-Asiatic (KE-AA) samples. (f) shows a meta-analysis of the combined P-values for each SNP over all subpopulations. G/C-14010 is highly significant, even after a Bonferroni correction (P=2.9×10⁻⁷). C/G-13907 and T/G-13915 are not significant after Bonferronni correction (P=0.001 and P=0.002, respectively).

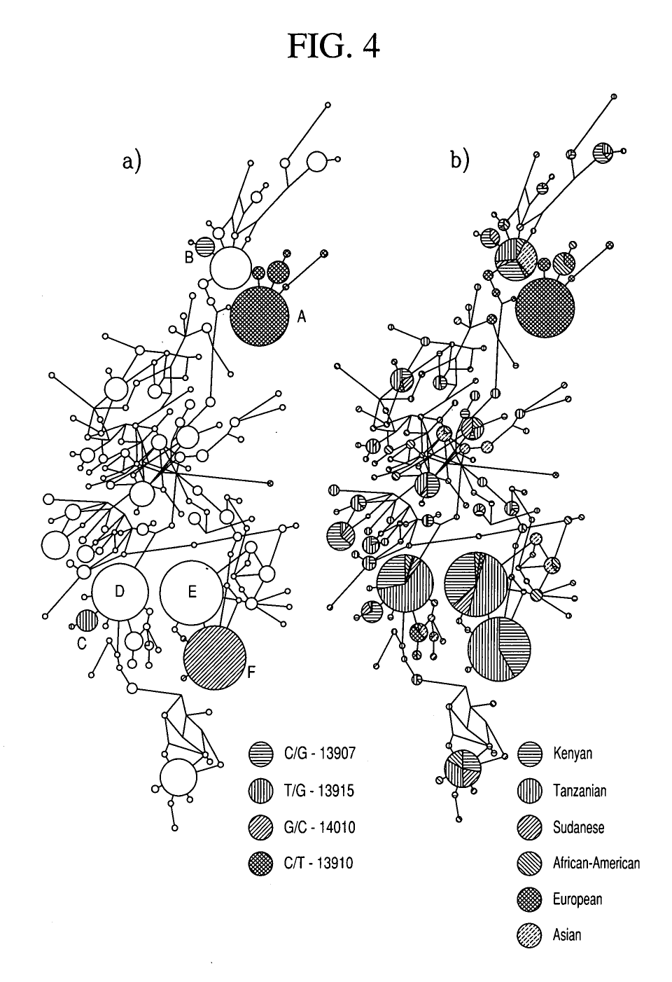

FIG. 4 shows haplotype networks consisting of 55 SNPs spanning a 98 kb region encompassing LCT and MCM6. In FIG. 4, (a) shows haplotypes with a T allele at −13910 are indicated by hatched lines, with a G allele at −13907 are indicated by horizontal lines, with a C allele at −14010 are indicated by diagonal lines, and with a G allele at −13915 are indicated by vertical lines. The arrow points to the inferred ancestral state haplotype. In FIG. 4, (b) shows a network analysis of LCT/MCM6 haplotypes indicating frequencies in the current data set, and in Europeans, Asians, and African Americans previously genotyped by Berseglieri et al. Bersaglieri, T. et al., Genetic signatures of strong recent positive selection at the lactase gene, Am J Hum Genet 74, 1111-20 (2004).

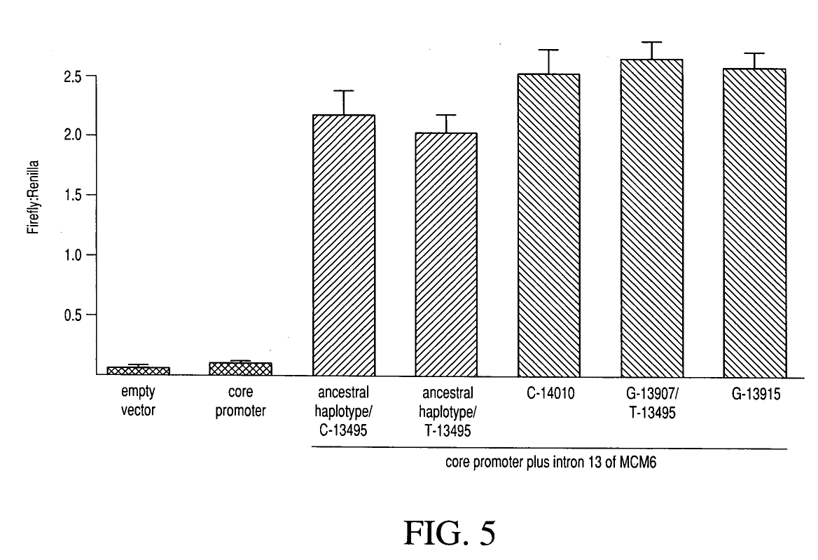

FIG. 5 shows a luciferase assay of LCT promoter and MCM6 introns. As a control, cells were transfected with the promoter-less pGL3-basic vector (Empty Vector). Basal levels of expression were assessed using a pGL3-basic vector with 3 kb of the 5′ flanking region of LCT (Core Promoter). Five different haplotypes of the MCM 6 intron 13 were inserted upstream of the core promoter that differed at the following sites: (1) a haplotype that is ancestral for the three LP-associated SNPs, with a C at position −13495; (2) a haplotype that is ancestral for the three LP-associated SNPs, with a T at position −13495; (3) a haplotype that differs from (1) only at C-14010; (4) a haplotype that differs from (1) at G-13907/T-13495 and from (2) only at G-13907; and (5) a haplotype that differs from (1) only at G-13915. Expression levels are reported as ratios of Firefly to Renilla and error bars represent 95% confidence intervals. The differences between the core promoter alone and all five MCM 6 intronic constructs, as well as between the three derived vs. two ancestral haplotypes were significant (p<0.0008, paired t-tests). There was no significant difference in expression levels between the empty vector and the core promoter, between the two ancestral haplotypes (with and without the T-13495 allele), or between the three derived haplotypes. The construct with ancestral LP-associated alleles that differed at T-13495 served as an internal control for the expression differences for the G-13907/T-13495 allele, indicating that only the G-13907 allele results in increased gene expression.

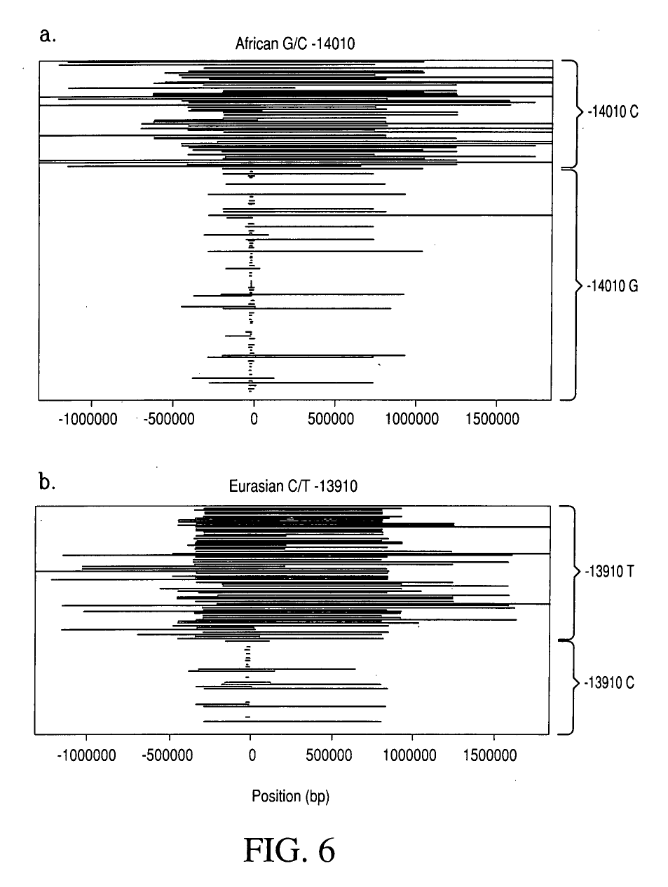

FIG. 6 shows a comparison of tracts of homozygous genotypes flanking the lactase persistence associated SNPs. In FIG. 6, (a) shows Kenyan and Tanzanian C-14010 lactase persistent and non-persistent G-14010 homozygosity tracts. In FIG. 6, (b) shows European and Asian T-13910 lactase persistent and C-13910 non-persistent homozygosity tracts, based on the data from Bersaglieri et al. Bersaglieri, T. et al., Genetic signatures of strong recent positive selection at the lactase gene, Am J Hum Genet 74, 1111-20 (2004). Positions are relative to the start codon of LCT. Note that some tracks are too short to be visible as plotted.

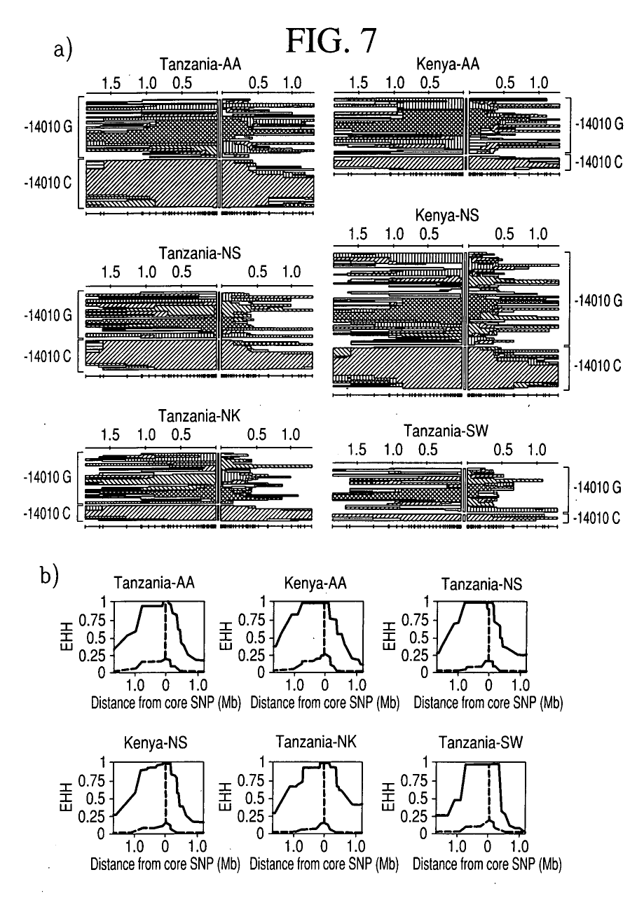

FIG. 7 shows plots of the extent and decay of haplotype homozygosity in the region surrounding the C-14010 allele. In FIG. 7, (a) shows the decay of haplotypes for the C-14010 allele in African subpopulations. Horizontal lines are haplotypes; SNP positions are marked below the haplotype plot. These plots are divided into two parts: the upper portion of the plot displays haplotypes with the ancestral G allele at site −14010 allele whereas the lower portion displays haplotypes with the derived C allele at −14010. For a given SNP, adjacent haplotypes with the same color carry identical genotypes everywhere between that SNP and the central (selected) site. The left- and right-hand sides are sorted separately. Haplotypes are no longer plotted beyond the points at which they become unique. Note the large extent of haplotype homozygosity surrounding the C-14010 allele (indicated by diagonal lines) extending as far as 2.9 Mbp in individual populations, which is consistent with the action of selection rapidly increasing the frequency of chromosomes with the C-14010 allele. In FIG. 7, (b) shows the decay of extended haplotype homozygosity for the C-14010 allele in African subpopulations over physical distance. In each case, the decay of haplotype homozygosity for the ancestral allele (shown in solid line) occurs much more quickly than for the derived allele (shown in dashed line). This is the expectation for strong positive selection acting on haplotypes containing this derived allele. AA denotes populations in the Afro-Asiatic language family. NK indicates Niger-Kordofanian, NS indicates Nilo-Saharan and SW indicates the Sandawe.

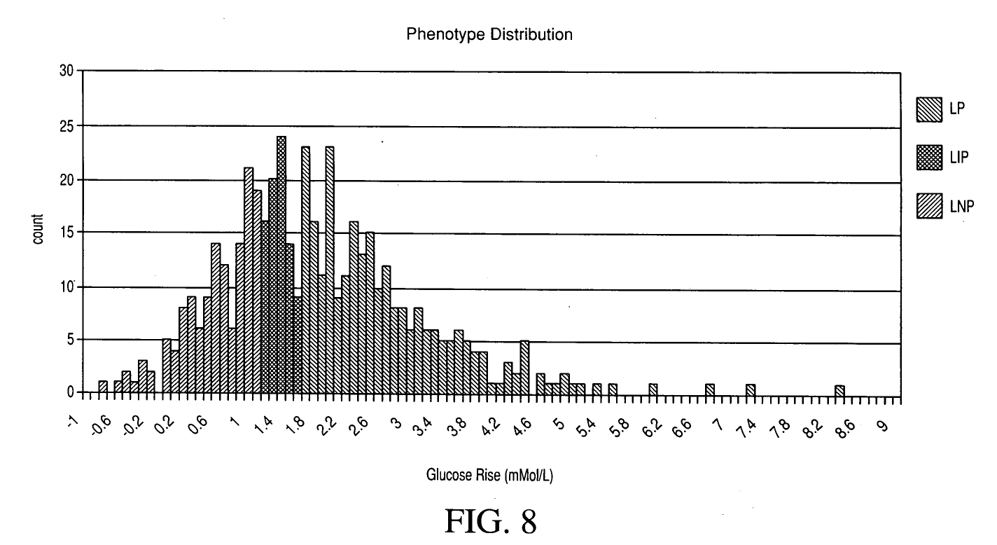

FIG. 8 shows the distribution of phenotype values for a pooled African dataset. In FIG. 8, values of LP>1.7 mMol/L glucose rise, 1.7 mMol/L>LIP>1.1 mMol/L, LNP<1.1 mMol/L are indicated by left diagonal, hatched, and right diaganol lines, respectively.

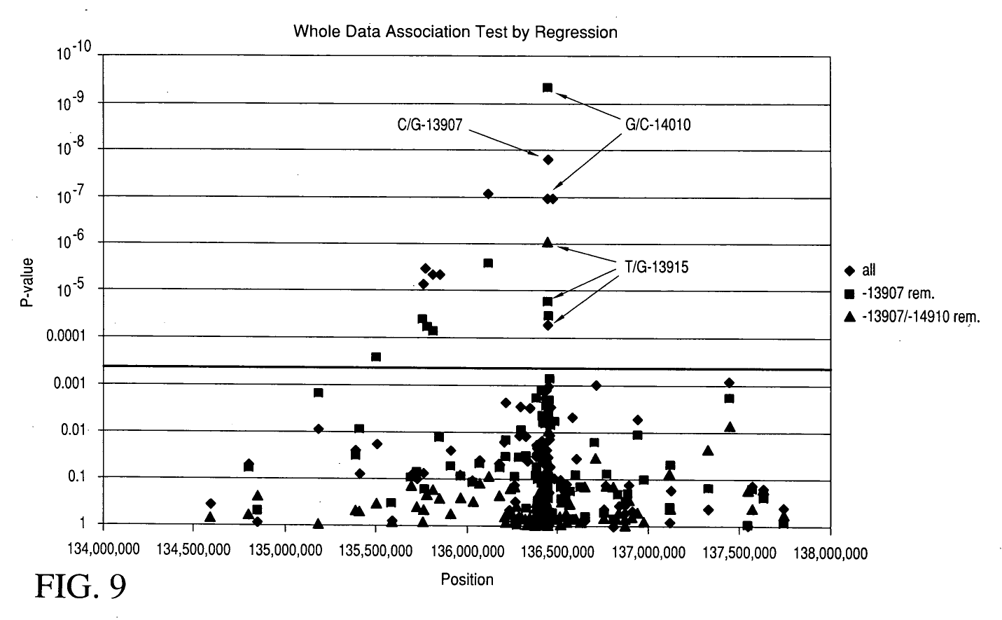

FIG. 9 shows linear regression based tests of association for each polymorphic SNP over a pooled dataset. The dark line denotes the significance level after a Bonferroni correction for the total number of SNPs tested (123). Although all three candidate SNPs are significantly associated with phenotype in the pooled populations (r²=0.067, P=1.06×10⁻⁷ for G/C-14010; r²=0.034, P=5.15×10⁻⁵ for T/G-13915; r²=0.067, P=1.63×10⁻⁸ for C/G-13907), C/G-13907 is the single most significant SNP in the pooled dataset, G/C-14010 is the most significant SNP after removal of individuals with at least one G or missing data at −13907, and T/G-13915 is the most significant SNP after removal of individuals with at least one G-13907 and/or C-14010 allele.

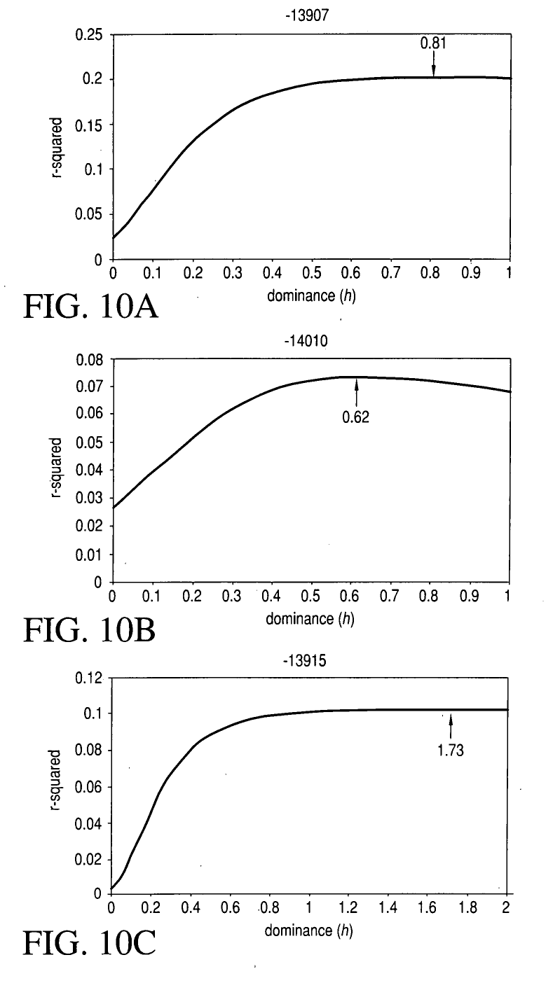

FIG. 10 shows an estimation of the degree of dominance for G/C-14010, T/G-13915 and C/G-13907. A linear regression is used and the phenotypes of the heterozygous individuals are adjusted along the x-axis between the two homozygous SNPs. The measure of fit, r-squared, was recorded at each position. Individuals that had at least one C at G/C-14010 were removed when plotting the results for C/G-13907 (a), individuals that had at least one G at C/G-13907 were removed when plotting the results for G/C-14010 (b) and individuals with at least one G at C/G-13907 or C at G/C-14010 were removed when plotting the results for T/G-13915 (c). C/G-13907 has a best fit value when the heterozygotes are at a position of 0.81 (a), but this value is barely better than complete dominance (i.e. a dominance value of 1). G/C-14010 has a more intermediate value of best fit at a dominance value of 0.62 (c). T/G-13915 has a best fit value consistent with overdominance, h=1.73; however, like C/G-13907, this value is barely better than a dominance value of 1.

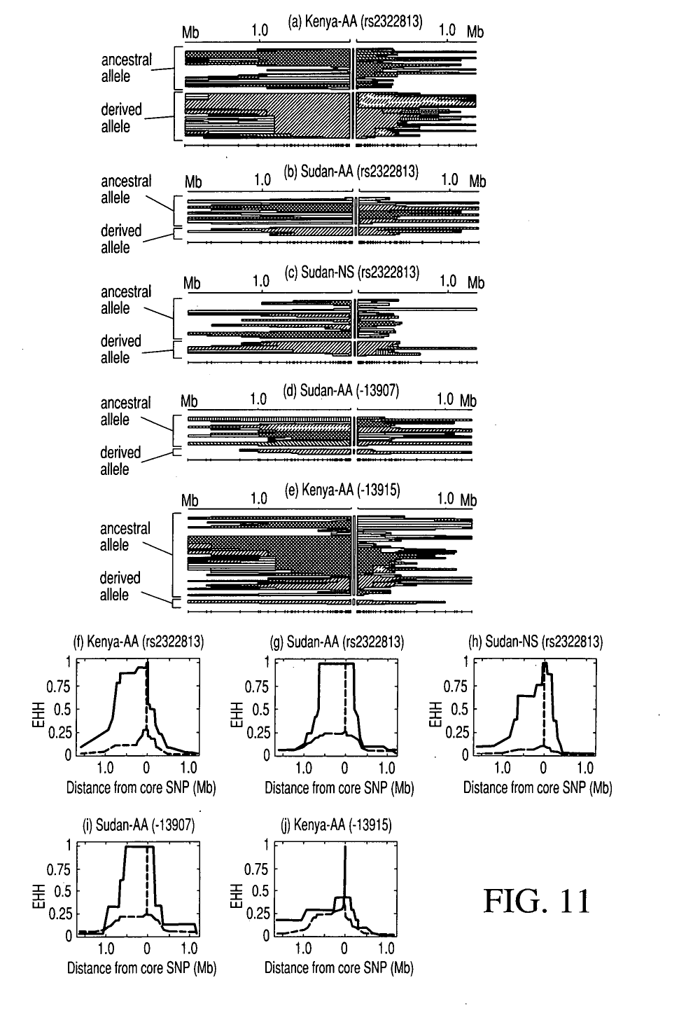

FIG. 11 shows plots of the extent and decay of haplotype homozygosity in the region surrounding the G-r2322813, G-13907 and G-13915 alleles. (a-c) Decay of haplotypes for the G-rs2322813 allele in Kenya AA, Sudan NS, and Sudan AA African subpopulations. Horizontal lines are haplotypes; SNP positions are marked below the haplotype plot. We assume that “ancestral alleles” are the most common allele. For a given SNP, adjacent haplotypes with the same pattern carry identical genotypes everywhere between that SNP and the central (selected) site. The left and right-hand sides are sorted separately. Haplotypes are no longer plotted beyond the points at which they become unique. (d) Decay of haplotypes for the G-13907 allele in the Sudan AA Beja population. (e) Decay of haplotypes for the G-13915 allele in the Kenyan AA population. (f-j) Decay of extended haplotype homozygosity for the G-rs2322813, G-13907, and G-13915 alleles (shown in solid lines) relative to the ancestral alleles (shown in dashed lines) over physical distance in the same populations as above.

By way of example, without limitation, exemplary embodiments of the present invention may be further illustrated with reference to the investigations discussed below.

Frequency of Lactase Persistence in East African Populations

In connection with the various exemplary embodiments of the present invention, the frequency of lactase persistence in East African populations has been investigated.

For example, the Classification of Lactase Persistence (LP), Lactase Intermediate Persistence (LIP) and Lactase Non-Persistence (LNP) was determined by examining the maximum rise in blood glucose levels following administration of 50 g of lactose using an LTT test²¹ in 470 individuals from 43 ethnic groups originating from Tanzania, Kenya, and Sudan. These populations speak languages belonging to the four major language families present in Africa (Afro-Asiatic—AA, Nilo-Saharan—NS, Niger-Kordofanian—NK, and Khoisan—KS) and practice a wide range of subsistence patterns, as illustrated by Table 2 and FIG. 2.

TABLE 2

| Region [symbol] | | | | | | | | | | | |
|---|---|---|---|---|---|---|---|---|---|---|---|
| Language | | | | Lactase | Lactase | | | | | | |
| Family [symbol] | | Pheno- | Lactase | [symbol] mediate | Non- | Geno- | −14010 | −13507 | −13910 | −13915 | −22016 |
| Population | Subsistence | typed | Persistant | Persistant | Persistant | typed | G % | G % | T % | G % | A % |
| Kenya | | | | | | | | | | | |
| Afro- [symbol] | | | | | | | | | | | |
| [symbol] | Agro [symbol] | [symbol] | [symbol] | 2 | 3 | [symbol] | [symbol] | 0.0 | 0.0 | 0.0 | 0.0 |
| [symbol] | [symbol] | [symbol] | [symbol] | 1 | 2 | [symbol] | 12.5 | 12.5 | 0.0 | 18.8 | 0.0 |
| El [symbol] | Fishing/Pastoralist | 0 | 4 | 3 | 2 | 9 | 11.1 | 0.0 | 0.0 | 0.0 | 0.0 |
| [symbol] | Pastoralist | [symbol] | [symbol] | 1 | 0 | 9 | 0.0 | 11.1 | 0.0 | 27.0 | 0.0 |
| [symbol] | Agro- [symbol] | [symbol] | 2 | 2 | 2 | [symbol] | [symbol] | 0.0 | 0.0 | 8.3 | 0.0 |
| [symbol] | Pastoralist | 6 | 6 | 1 | 2 | 8 | 12.5 | 8.3 | 0.0 | 12.6 | 0.0 |
| [symbol] | Pastoralist | 1 | 1 | 0 | 0 | 1 | 0.0 | 0.0 | 0.0 | 50.0 | 0.0 |
| [symbol] | Hunter [symbol] gatherer | 1 | 0 | 0 | 1 | 1 | 0.0 | 0.0 | 0.0 | 0.0 | 0.0 |
| [symbol] | Hunter | 14 | 6 | 5 | 1 | 14 | 53.6 | 4.2 | 0.0 | 0.0 | 0.0 |
| | gatherers [symbol] Pastoralist | | | | | | | | | | |
| Total | | 64 | 86 | 15 | 13 | [symbol] | 18.3 | [symbol] | 0.0 | 9.4 | 0.0 |
| [symbol] | | | | | | | | | | | |
| [symbol] | Pastoralist | 32 | 23 | 6 | 3 | 32 | 57.6 | 3.1 | 0.0 | 0.0 | 0.0 |
| [symbol] | Agro-pastoralist | 6 | 3 | 1 | 2 | 7 | 35.7 | 0.0 | 0.0 | 7.1 | 0.0 |
| [symbol] | Agro-pastoralist | 4 | [symbol] | 2 | 2 | 4 | 23.0 | 0.0 | 0.0 | 0.0 | 0.0 |
| [symbol] | Agro- | 11 | 6 | 0 | [symbol] | 11 | 26.4 | 0.0 | 0.0 | 0.0 | 0.0 |
| | [symbol] | | | | | | | | | | |
| | gatherers | | | | | | | | | | |
| [symbol] | [symbol] | 14 | 6 | 4 | [symbol] | 14 | 23.6 | 0.0 | 0.0 | 3.5 | 0.0 |
| [symbol] | Agro [symbol] | 6 | [symbol] | 2 | 1 | 6 | 10.7 | 0.0 | 0.0 | 0.0 | 0.0 |
| [symbol] | [symbol] | 9 | 0 | 0 | 1 | 0 | [symbol] | 6.3 | 0.0 | 5.6 | 0.0 |
| [symbol] | Agro- | 10 | 2 | 4 | 10 | 10 | 6.3 | 0.0 | 0.0 | 0.0 | 0.0 |
| | [symbol] | | | | | | | | | | |
| | gatherers | | | | | | | | | | |
| [symbol] | [symbol] | 15 | [symbol] | [symbol] | 3 | [symbol] | 0.0 | 0.0 | 0.0 | 0.0 | |
| [symbol] | [symbol] | 12 | 4 | 4 | 4 | 13 | 20.8 | 0.0 | 0.0 | 0.0 | 0.0 |
| Total | | 125 | [symbol] | [symbol] | 35 | 123 | 31.5 | 1.2 | 0.0 | 1.2 | 0.0 |
| Niger [symbol] | | | | | | | | | | | |
| [symbol] | [symbol] | 2 | 1 | 0 | 1 | 2 | 75.0 | 0.0 | 0.0 | 0.0 | 0.0 |
| Kenya Total | | 192 | 100 | 43 | 49 | 194 | 27.0 | 2.4 | 0.0 | 3.9 | 0.0 |
| Sudan | | | | | | | | | | | |
| Afro [symbol] | | | | | | | | | | | |
| [symbol] | Pastoralist | 6 | 6 | 0 | 0 | 6 | 0.0 | 25.0 | 0.0 | 18.7 | 0.0 |
| [symbol] | Pastoralist | 11 | [symbol] | 0 | 2 | 11 | 0.0 | 18.2 | 0.0 | 9.1 | 0.0 |
| Total | | 17 | 15 | 0 | 2 | 17 | 0.0 | 20.6 | 0.0 | [symbol] | 0.0 |
| [symbol] | | | | | | | | | | | |
| [symbol] | Pastoralist | 9 | 6 | 2 | 1 | 9 | 0.0 | 0.0 | 0.0 | 0.0 | 0.0 |
| [symbol] | Agro-pastoralist | 1 | 1 | 0 | 0 | 1 | 0.0 | 0.0 | 0.0 | 0.0 | 0.0 |
| [symbol] | Agro-pastoralist | 1 | 0 | 0 | 1 | 1 | 0.0 | 0.0 | 0.0 | 0.0 | 0.0 |
| [symbol] | Agro-pastoralist | 1 | 1 | 0 | 0 | 1 | 0.0 | 0.0 | 0.0 | 0.0 | 0.0 |
| [symbol] | Pastoralist | 4 | 2 | 2 | 0 | 6 | 0.0 | 0.0 | 0.0 | 0.0 | 0.0 |
| [symbol] | Agro-pastoralist | 2 | 1 | 0 | 1 | 2 | 0.0 | 0.0 | 0.0 | 0.0 | 0.0 |
| [symbol] | Pastoralist | [symbol] | 3 | 4 | 1 | 6 | 0.0 | 0.0 | 0.0 | 0.0 | 0.0 |
| Total | | 26 | 14 | 8 | 4 | 27 | 0.0 | 0.0 | 0.0 | 0.0 | 0.0 |
| Sudan Total | | 43 | [symbol] | 8 | 6 | 44 | 0.0 | 8.0 | 0.0 | 4.5 | 0.0 |
| Tanzania | | | | | | | | | | | |
| Afro-Asiatic | | | | | | | | | | | |
| Burunge | Agro-pastoralist | 18 | 6 | 2 | 10 | 18 | 30.2 | 0.0 | 0.0 | 0.0 | 0.0 |
| [symbol] | Agro-pastoralist | 21 | 18 | 2 | 1 | [symbol] | 67.7 | 0.0 | 0.0 | 0.0 | 0.0 |
| [symbol] | Agro-pastoralist | 30 | 10 | 7 | 13 | 30 | 31.0 | 0.0 | 0.0 | 0.0 | 0.0 |
| [symbol] | Agro-pastoralist | 12 | 1 | [symbol] | 6 | 12 | 55.0 | 0.0 | 0.0 | 0.0 | 0.0 |
| Total | | 81 | 35 | 18 | 30 | 99 | 45.6 | 0.0 | 0.0 | 0.0 | 0.0 |
| [symbol] | | | | | | | | | | | |
| Akia | Hunter-gatherers/ | 14 | 6 | 3 | 5 | 14 | 25.0 | 0.0 | 0.0 | 0.3 | 3.6 |
| | pastoralist | | | | | | | | | | |
| [symbol] | Pastoralist | 4 | 0 | 3 | 1 | 4 | 62.5 | 0.0 | 0.0 | 0.0 | 0.0 |
| [symbol] | Hunter-gatherers/ | 10 | 4 | 4 | 2 | 10 | 40.0 | 0.0 | 0.0 | 0.0 | 0.0 |
| | Pastoralist | | | | | | | | | | |
| [symbol] | Pastoralist | 17 | 10 | 2 | 5 | 19 | 44.7 | 0.0 | 0.0 | 0.0 | 0.0 |
| Total | | 45 | 20 | 12 | 13 | 47 | 39.4 | 0.0 | 0.0 | 0.0 | 1.1 |
| [symbol] | | | | | | | | | | | |
| [symbol] | Agro-pastoralist | 13 | 4 | 5 | 4 | 13 | 26.9 | 0.0 | 0.0 | 3.8 | 0.0 |
| Paro | Agro-pastoralist | 10 | 0 | 2 | 2 | 10 | 10.0 | 0.0 | 0.0 | 0.0 | 0.0 |
| Kangi | Agro-pastoralist | 35 | 17 | 0 | 9 | [symbol] | 27.1 | 0.0 | 0.0 | 0.0 | 0.0 |
| Sam [symbol] | Agro-pastoralist | 3 | 0 | 1 | 2 | 3 | 0.0 | 0.0 | 0.0 | 0.0 | 0.0 |
| Total | | 61 | 27 | 17 | 17 | 65 | 23.0 | 0.0 | 0.0 | 0.0 | 0.0 |
| [symbol] | | | | | | | | | | | |
| [symbol] | Hunter-gatherer | 18 | 8 | 3 | 6 | 18 | 0.0 | 0.0 | 0.0 | 0.0 | 0.0 |
| [symbol] | Hunter-gatherer | 30 | 8 | 7 | 26 | 21 | 13.3 | 0.0 | 0.0 | 0.0 | 0.0 |
| Total | | 48 | 17 | 10 | 21 | 49 | 6.3 | 0.0 | 0.0 | 0.0 | 0.0 |
| Tanzania | | 235 | [symbol] | 55 | 81 | 256 | 31.0 | 0.0 | 0.0 | 0.2 | 0.2 |
| Total | | | | | | | | | | | |
| East Africa | | 470 | 220 | 103 | 136 | 494 | 27.4 | 1.6 | 0.0 | 2.0 | 0.1 |
| Total | | | | | | | | | | | |
| [symbol] | | | | | | | | | | | |
| CEPH- | | — | — | — | — | 92 | — | — | 61.5 | — | 81.7 |
| European | | | | | | | | | | | |
| African | | — | — | — | — | [symbol] | — | — | 14.0 | — | 13.3 |
| American | | | | | | | | | | | |
| Asian | | — | — | — | — | 33 | — | — | 0.0 | — | 0.0 |

[symbol]

indicates data missing or illegible when filed

Because genetic substructure can result in false genotype/phenotype associations (Pritchard et al., Association mapping in structured populations, Am J Hum Genet 67, 170-81 (2000)), data were analyzed from samples separated by geographic region and language family, with the exception of the Sandawe and Hadza (both click-speaking Khoisan) who were analyzed independently. See FIG. 2. These groupings were made in order to minimize population structure, based on a global analysis of ˜1200 unlinked nuclear markers (Reed and Tishkoff, unpublished data). The frequency of LP is highest in the AA-speaking Beja pastoralist population from Sudan (88%) and lowest in the KS-speaking Sandawe hunter-gatherer population from Tanzania (26%). See FIG. 2(a) and Table 2.
Identification of SNPs Associated with Lactase Persistence in Africans

In connection with the various exemplary embodiments of the present invention, SNP's associated with lactase persistence in Africans have been investigated.

For example, to identify SNPs associated with regulation of the LP trait, 40 LNP and 69 LP individuals were sequenced at the extremes of the phenotype distribution (FIG. 8) for 3,314 bp of intron 13 and 1,761 bp of intron 9 of MCM6 (FIGS. 1 c and d). A novel SNP, G/C-14010, showed a significant association with the LP trait in Kenyans (n=53) and Tanzanians (n=31) (Ω²=14.4, d.f.=2, P=0.0007 and Ω²=10.9, d.f.=2, P=0.0043, respectively. (FIG. 1d). A second novel SNP, T/G-13915, was significantly associated with LP in Kenyans (n=53, d.f.=1, Ω^{2=4.70}, P=0.0302), and a third novel SNP, C/G-13907, was marginally significantly associated with LP in the Beja population from Northern Sudan. (n=11, d.f.=1, Ω2=2.93, P=0.0869). (FIG. 1d). Sequencing of these regions in a panel of great apes indicated that the C-14010, G-13915, and G-13907 alleles are derived.

In order to determine regional haplotype structure and further characterize the frequency and degree of association of these alleles, 123 SNPs (including G/C-14010, T/G-13915, and C/G-13907) were genotyped across a 3 Mbp region flanking the MCM6 and LCT genes in the full set of 470 individuals with reliable phenotype data and in 24 additional individuals. (FIG. 1a; Table 3). The genotype/phenotype distribution and Ω² tests of association for the three candidate SNPs and data partitioned according to LP, LIP, and LNP classification in major geographic regions are shown in FIG. 3 a-d. Additionally, a linear-regression approach was used, which accounts for the continuous phenotype distribution, to test for an association between all 123 SNPs and rise in blood glucose following digestion of lactose. Reed et al., Evidence of susceptibility and resistance to cryptic X-linked meiotic drive in natural populations of Drosophila melanogaster, Evolution Int J Org Evolution 59, 1280-91 (2005), Cheung et al., Mapping determinants of human gene expression by regional and genome-wide association, Nature 437, 1365-9 (2005). Results from individual populations and from a meta-analysis of the combined P-values for all subpopulations are shown in FIG. 3e and FIG. 3f, respectively. G/C-14010 is the most significantly associated SNP in the Kenyan NS and Tanzanian AA populations (r²=0.19 and 0.16, and P=2.67×10⁻⁷ and 2.79×10⁻⁴, respectively) as well as over all populations combined in the meta-analysis (P=2.9×10⁻⁷). Although C/G-13907 and T/G-13915 are associated with the phenotype, this association was not statistically significant after Bonferroni correction in either the individual populations or in the meta-analysis. (FIG. 3e-f). It is pointed out that the C-14010, G-13907, and G-13915 alleles in Africans exist on haplotype backgrounds that are divergent from each other and from the European T-13010 haplotype background (FIG. 4).

Table 3

TABLE 3
Genotyped SNP identifications and locations.

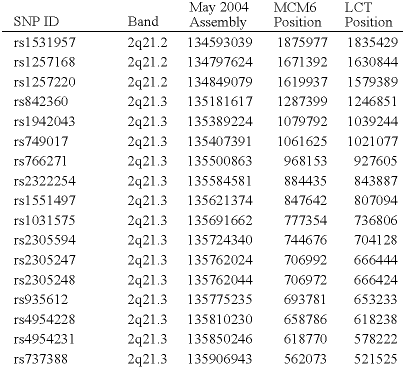

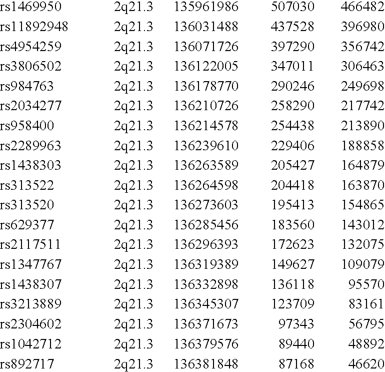

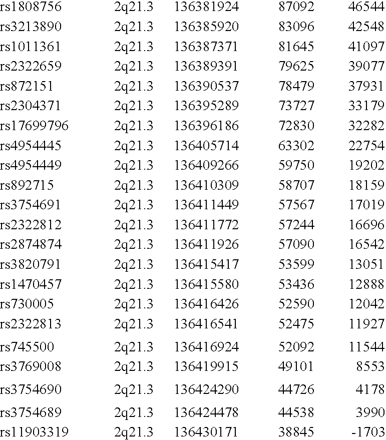

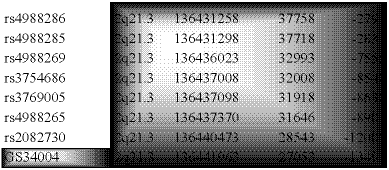

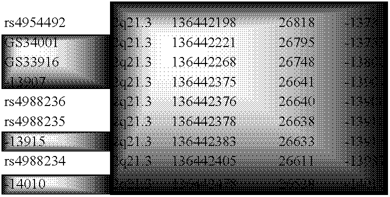

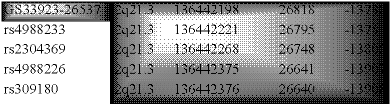

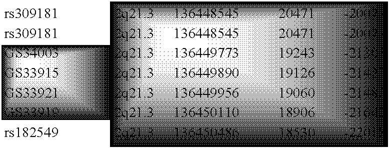

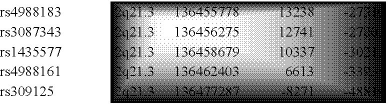

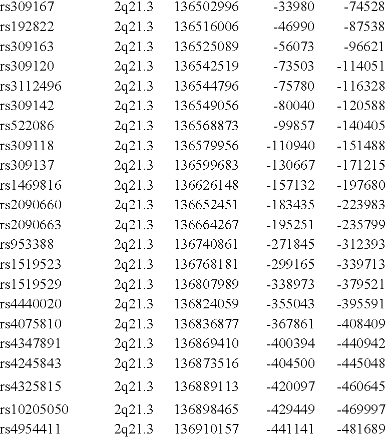

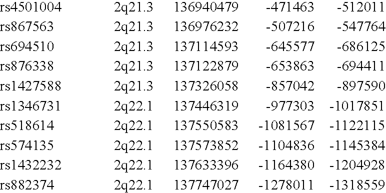

LEGEND

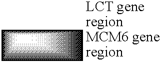

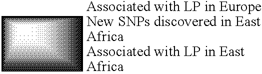

Based on ANOVA analysis of the phenotypes for each of the six classes of observed compound G/C-14010, T/G-13915, and C/G-13907 genotypes, ˜20% of the total phenotypic variation is accounted for by the genotypes in the pooled sample, suggesting that there may be environmental and/or measurement factors and possibly unidentified genetic factors, influencing the LTT phenotype in this dataset.

Frequencies of G/C-14010, T/G-13915, and C/G-13907 in African Populations

In connection with the various exemplary embodiments of the present invention, the frequencies of G/C-14010, T/G-13915, and C/G-13907 in African populations was investigated.

For example, genotype frequencies for G/C-14010, T/G-13915, and C/G-13907 are shown in FIG. 2b, whereas Table 2 shows allele frequencies for these SNPs as well as the European LP-associated SNPs C/T-13910 and G/A-22018. The T-13910 allele is absent in all of the African populations tested and the A-22018 allele was observed in a single heterozygous Akie individual from Tanzania. The C-14010 allele is common in NS populations from Tanzania (39%) and Kenya (32%) and in AA populations of Tanzania (46%), but occurs at a lower frequency in the Sandawe (13%) and AA Kenyan (18%) populations, and is absent in the NS Sudanese and Hadza populations. (FIG. 2b; Table 2). The C-13907 and G-13915 alleles are at ≧5% frequency only in the AA Beja (21% and 12%, respectively) and in the AA Kenyan (5% and 9%, respectively) populations.

Effect of C-14010, G-13915, and G-13907 on Transcript Expression from the LCT Promoter

In connection with the various exemplary embodiments of the present invention, the effect of C-14010, G-13915, and G-13907 on transcript expression from the LCT promoter was investigated.

For example, in order to test whether the C-14010, G-13915, and G-13907 mutations affect mRNA expression from the LCT core promoter, we transfected the human intestinal cell line Caco-2 with luciferase expression vectors driven by the basal 3 kb promoter alone or the promoter fused to one of five haplotypes of the 2 kb MCM6 intron 13 region; a haplotype with ancestral alleles at the three candidate SNPs (G-14010, T-13915, C-13907), two haplotypes which differed only at the derived C-14010 or G-13915 alleles, a haplotype that differed at the derived G-13907 allele as well as a linked T-13495 allele, and a haplotype that has the ancestral LP-associated alleles, with a T at position −13945 (to control for the effect of this mutation). Differences in luciferase expression between the basal 3 kb LCT core promoter and the promoter plus any of the five MCM6 intron sequence constructs were highly significant (paired t-test, p<0.001), resulting in a >20 fold increase in expression, as compared to the core promoter alone (FIG. 5).

Differences in expression were also observed between the five MCM6 intron 13 haplotypes that were functionally tested using the DLR assay (FIG. 5). The C-14010, G-13915, and G-13907 derived haplotypes consistently drove higher expression (from ˜18-30%) compared to the haplotypes with the ancestral alleles. There was no statistically significant difference in expression between the constructs with the C-14010, G-13907/T-13495, and G-13915 alleles.

Evidence for Positive Selection of the C-14010 Allele

In connection with the various exemplary embodiments of the present invention, evidence for positive selection of the C-14010 allele was investigated.

For example, it is hypothesized that if a mutation provides a large enough benefit to its carriers (in this case, the ability to digest milk as adults), resulting in more viable offspring, it is expected to rise rapidly to high frequency in the population, together with linked variants (i.e. genetic hitchhiking). Maynard et al., The hitchhiking effect of a favourable gene, Genetical Research 23, 23-35 (1974). Under neutrality, one expects common mutations to be older and to have lower levels of LD with flanking markers. In contrast, one of the genetic signatures of an incomplete selective sweep is a region of extensive LD (extended haplotype homozygosity, “EHH”) and low variation on high frequency chromosomes with the derived beneficial mutation relative to chromosomes with the ancestral allele. Voight et al., A map of recent positive selection in the human genome, PLoS Biol 4, e72 (2006); Sabeti et al., Detecting recent positive selection in the human genome from haplotype structure, Nature 419, 832-7 (2002). Over time, this pattern will degrade due to recombination and newly occurring mutations. Thus, by measuring the frequency of the haplotype and extent of LD in the region, it is possible to estimate the age and strength of a beneficial mutation.

In order to visually assess the evidence for selection on chromosomes with the C-14010 mutation, plots were constructed depicting EHH for ancestral (G) and derived (C) alleles using both unphased data (FIG. 6), as well as phase inferred data (FIG. 7). For the unphased data, continuous homozygosity was plotted at each of the 123 genotyped SNPs for individuals homozygous for the ancestral (G/G-14010) and derived (C/C-14010) alleles (FIG. 6a). For comparison, EHH was plotted for the 101 SNPs genotyped in Eurasians by Bersaglieri et al. for individuals homozygous for the ancestral (C/C-13010) and derived (T/T-13010) LP-associated alleles. (FIG. 6b). Bersaglieri, T. et al., Genetic signatures of strong recent positive selection at the lactase gene, Am J Hum Genet 74, 1111-20 (2004). The average homozygous tract length in C/C-14010 homozygotes (N=51) is 1.8 Mbp (maximum of 3.15 Mbp), compared to 1,800 bp in G/G-14010 homozyogotes (N=228). In Eurasians, the average homozygous tract length in T/T-13010 homozygotes (N=61) is 1.4 Mbp (maximum of 2.1 Mbp), compared to 1,900 bp in C/C-13010 homozygotes (N=38). A similar result is observed in the individual African populations using phase inferred data, with EHH extending as far as 2.18-2.90 Mbp (1.6-2.2 cM). (Table 1 and FIG. 7). Chromosomes with the G-13907 and G-13915 mutations exhibit EHH spanning ˜1.4 Mbp (0.56 cM) and 1.1 Mbp (0.37 cM), respectively. (FIG. 9).

The high frequency of the C-14010 allele and the remarkably long stretch of homozygosity extending >2 Mbp for haplotypes containing the C-14010 allele are consistent with the action of positive selection elevating this allele, and the surrounding linked variation, to high frequency. To test the neutrality of this SNP, a modification of the EHH test was used, the integrated haplotype score (iHS) (note that sample sizes for G-13915 and G-13907 alleles are too small for sufficient power with the iHS test). Sabeti et al., Detecting recent positive selection in the human genome from haplotype structure, Nature 419, 832-7 (2002); Voight et al., A map of recent positive selection in the human genome, PLoS Biol 4, e72 (2006). For most populations, the iHS score was statistically more extreme relative to iHS scores for data simulated under a neutral model with constant population size (p<0.002), as well as compared to data simulated under an assortment of demographic population expansion and contraction models. See Table 4. All populations had statistically more extreme scores relative to the empirical distribution of iHS scores observed in the Yoruban Hapmap data, for alleles at matching frequency (p<0.05) (Table 1). Furthermore, as predicted, the direction of the score was consistent with the action of positive selection on the LP-associated haplotype.

TABLE 1
EHH statistics and estimates of age of the C-14010 mutation and selection coefficients

| | | | | | | | | Dominant Model | | Additive Model | |
|---|---|---|---|---|---|---|---|---|---|---|---|
| Popu- | Sample | Frequency | | | | | | s | Age (y) | s | Age (y) |
| lation | Size (n) | (C-14010) | iHS | p-stimul | p-emp | Span (cM) | Span (Mb) | [95% CI] | [95% CI] | [95% CI] | [95% CI] |
| Kenya-AA | 64 | 0.18 | −0.79 | 0.204 | 0.043 | 2.17 | 2.73 | 0.07 | 2966 | 0.095 | 3764 |
| | | | | | | | | [0.022-0.142] | [1215-6827] | [0.033-0.1461] | [1970-8036] |
| Kenya-NS | 128 | 0.316 | −2.8 | 0.002 | 0.00013 | 1.64 | 2.27 | 0.035 | 6925 | 0.067 | 6167 |
| | | | | | | | | [0.008-0.080] | [2232-18496] | [0.020-0.137] | [2478-14785] |
| Tanzania- | 99 | 0.449 | −2.78 | <0.001 | 0.0012 | 2.02 | 2.53 | 0.053 | 5956 | 0.072 | 6591 |
| AA | | | | | | | | [0.018-0.130] | [1575-13054] | [0.024-0.138] | [2819-16072] |
| Tanzania- | 47 | 0.394 | −2.85 | <0.001 | 0.00059 | 2.07 | 2.78 | 0.07 | 3757 | 0.097 | 4358 |
| NS | | | | | | | | [0.023-0.143] | [1344-9087] | [0.040-0.145] | [2609-9476] |
| Tanzania- | 61 | 0.23 | −2.61 | <0.003 | 0.00032 | 2.22 | 2.9 | 0.077 | 2778 | 0.097 | 4075 |
| NK | | | | | | | | [0.026-0.142] | [1219-6049] | [0.036-0.148] | [2304-9533] |
| Tanzania- | 18 | 0.129 | −1.19 | 0.112 | 0.024 | 1.6 | 2.18 | 0.043 | 5717 | 0.06 | 6899 |
| SW | | | | | | | | [0.005-0.132] | [1296-17971] | [0.007-0.135] | [2050-23291] |
| European | 48 | 0.76 | −3.86 | <0.001 | N/A | 1.58 | 2.15 | 0.039 | 9323 | 0.069 | 7998 |
| | | | | | | | | [0.012-0.107] | [2231-19228] | [0.025-0.132] | [3466-18191] |

“iHS”: Standardized integrated Haplotype (iHS) Score for C-14010.
“p-simul”: p-value for the iHS score from simulations.
“p-emp”: empirical p-value for the iHS score using the observed iHS scores at the specified derived allele frequency for the Hapmap Yoruba sample.
“cM span”: assuming the position where the probability of haplotype identity is 0.25.
“s”: selection intensity (estimated from simulation), assuming an effective population size of 10,000.
The European data is from Bersaglieri et al. See Bersaglieri et al., Genetic signatures of strong recent positive selection at the lactase gene, Am J Hum Genet 74, 1111-20 (2004).

TABLE 4
Significance of iHS under assorted demographic models

| Demographic Model | Kenya-AA | Kenya-NS | Tanzania-AA | Tanzania-NS | Tanzania-NK | Tanzania-KS |
|---|---|---|---|---|---|---|
| Growth (Model 1) | 0.14 | <0.01 | <0.01 | <0.01 | <0.01 | 0.13 |
| Growth (Model 2) | 0.14 | <0.01 | <0.01 | <0.01 | <0.01 | 0.12 |
| Growth (Model 3) | 0.21 | <0.01 | <0.01 | <0.01 | <0.01 | 0.13 |
| Growth (Model 4) | 0.17 | <0.01 | <0.01 | <0.01 | <0.01 | 0.24 |
| Growth (Model 5) | 0.15 | <0.01 | <0.01 | <0.01 | <0.01 | 0.11 |
| Growth (Model 6) | 0.15 | <0.01 | <0.01 | <0.01 | <0.01 | 0.09 |
| Bottleneck (Model 1) | 0.14 | <0.01 | <0.01 | <0.01 | <0.01 | <0.01 |
| Bottleneck (Model 2) | 0.13 | <0.01 | <0.01 | <0.01 | <0.01 | <0.01 |
| Bottleneck (Model 3) | 0.02 | <0.01 | <0.01 | <0.01 | <0.01 | <0.01 |
| Bottleneck (Model 4) | 0.011 | <0.01 | <0.01 | <0.01 | <0.01 | <0.01 |
| Bottleneck (Model 5) | <0.01 | <0.01 | <0.01 | <0.01 | <0.01 | <0.01 |
| Bottleneck (Model 6) | <0.01 | <0.01 | <0.01 | <0.01 | <0.01 | 0.13 |
| Bottleneck (Model 7) | 0.011 | <0.01 | <0.01 | <0.01 | <0.01 | 0.11 |
| Bottleneck (Model 8) | <0.01 | <0.01 | <0.01 | <0.01 | <0.01 | <0.01 |
| Bottleneck (Model 9) | <0.01 | <0.01 | <0.01 | <0.01 | <0.01 | <0.01 |
| Bottleneck (Model 10) | <0.01 | <0.01 | <0.01 | <0.01 | <0.01 | <0.01 |
| Bottleneck (Model 11) | <0.01 | <0.01 | <0.01 | <0.01 | <0.01 | <0.01 |

### Growth Models

Exponential growth beginning at tₒₙₛₑₜ generations in the past at rate alpha: N_{F}=N_{A}*exp(tₒₙₛₑₜ*α). Various models were taken from Voight et al. (2005).

1: α=0, N_{A}=11156 [no growth]
2: α=0.00075, tₒₙₛₑₜ=1000, N_{A}=10659 [˜2× growth starting 25,000 years ago, approximate MLE for Hausa data based on Voight et al. (2005)]
3: α=0.01, tₒₙₛₑₜ=250, N_{A}=10860 [˜12× growth starting 6,250 years ago]
4: α=0.00025, tₒₙₛₑₜ=5000, N_{A}=8449 [˜4× growth starting 125,000 ya]
5: α=0.00075, tₒₙₛₑₜ=1000, N_{A}=12300 [same as 2, with upper confidence bound for N_{A} based on Voight et al. (2005)]
6: α=0.00075, tₒₙₛₑₜ=1000, N_{A}=9450 [same as 3, with lower confidence bound for N_{A} based on Voight et al. (2005)]

### Bottleneck Models

A population of ancestral size N_{A} experiences an instantaneous reduction in population size to b*N_{A}, which persists for t_{dur} generations. The population recovers to 1× (Models 1-5), 10× (Models 6-10), or 50× (Models 11 & 12) of the ancestral population size after the bottleneck.

Bottleneck, with recovery after the bottleneck to initial population size [N_{A}=10,659]

1: b=1.0 [no bottleneck]
2: b=0.1, t_{dur}=100, T=1600 [90% reduction in population size occurring 37,500 years ago lasting 2,500 years]
3: b=0.01, t_{dur}=100, T=1600 [99% reduction in population size occurring 37,500 years ago lasting 2,500 years]
4: b=0.01, t_{dur}=200, T=1600 [99% reduction in population size occurring 35,000 years ago lasting 5,000 years]
5: b=0.01, t_{dur}=400, T=1600 [99% reduction in population size occurring 30,000 years ago lasting 10,000 years]

Bottleneck, with 10× increase in original population size after the bottleneck [N_{A}=10,659]

6: b=0.01, t_{dur}=100, T=1600 [99% reduction in population size occurring 37,500 years ago lasting 2,500 years]
7: b=0.01, t_{dur}=200, T=1600 [99% reduction in population size occurring 35,000 years ago lasting 5,000 years]
8: b=0.01, t_{dur}=100, T=400 [99% reduction in population size occurring 7,500 years ago lasting 2,500 years]
9: b=0.01, t_{dur}=100, T=200 [99% reduction in population size occurring 2,500 years ago lasting 2,500 years]

Bottleneck, with 50× increase in pop size after the bottleneck [N_{A}=10,659]

10: b=0.01, t_{dur}=50, T=200 [99% reduction in population size occurring 3,750 ya lasting 1,250 years]
11: b=0.005, t_{dur}=50, T=200 [99.5% reduction in population size occurring 3,750 ya lasting 1,250 years]

Age of the LP Associated Mutations and Estimates of Selection Coefficients

In connection with the various exemplary embodiments of the present invention, the age of the LP associated mutations and estimates of selection coefficients were investigated.

For example, the age of the C-14010 allele was estimated using coalescent simulations under a model incorporating selection and recombination. Spencer et al., SelSim: a program to simulate population genetic data with natural selection and recombination, Bioinformatics 20, 3673-5 (2004). The simulations assumed either an additive (h=0.5) or dominant (h=1) model for fitness, and were designed to match several aspects of the data including SNP ascertainment and density, allele frequency, sample size, recombination profile, and phase uncertainty. Voight et al., A map of recent positive selection in the human genome, PLoS Biol 4, e72 (2006). Selection intensity and ages were estimated by matching simulated data to the observed cM span and the observed frequency of the derived allele in each population. Estimates of these values are presented in Table 1, and demonstrate extremely recent (within the last ˜3-7 ky, CI 1.2-23.2 ky) and strong (s=0.04-0.097, CI 0.01-0.15), positive selection in many African populations.

Evidence that G/C-14010, T/G-13915, and C/G-13907 Regulate LCT Gene Expression

In connection with the various exemplary embodiments of the present invention, evidence that G/C-14010, T/G-13915, and C/G-13907 regulate LCT gene expression is investigated and discussed. The data indicates that G/C-14010 regulates LCT gene expression.

First, this SNP shows significant statistical association with the LTT phenotype in Kenyan and Tanzanian populations (FIG. 3). Although most individuals with a C-14010 allele have moderate to high increases in blood glucose (mean of 2.04 and 2.45 mM/L in heterozygotes and homozygotes, respectively; (FIG. 2b), many individuals who are homozygous for the ancestral G-14010 allele are also classified as LIP or LP (FIG. 3), likely due to genetic heterogeneity of this trait, as discussed further below. Additionally, there is likely to be phenotype measurement error due to working in field conditions and to the relative insensitivity of the LTT test (see methods). Also, individuals with the C-14010 allele may be classified as LNP if they have had damage to intestinal cells caused by infectious disease. Arola, Diagnosis of hypolactasia and lactose malabsorption, Scand J Gastroenterol Suppl 202, 26-35 (1994).

Second, extensive LD was observed on chromosomes with the C-14010 mutation, with haplotype homozygosity extending >2 Mbp. (FIGS. 6 and 7). Of the 123 SNPs genotyped, high LD (D′>0.9, LOD scores≧2) extends the farthest distance for SNP G/C-14010 (FIG. 10) and is inconsistent with demographic models that incorporate even extreme bottlenecks. In fact, this region of haplotype identity, spanning 2.18-2.9 Mbp (1.6-2.2 cM), is more extensive than any span of identity previously reported in the genome based on Hapmap data from global populations. The International HapMap Consortium, A haplotype map of the human genome, Nature 437, 1299-1320 (2005); Voight et al., A map of recent positive selection in the human genome, PLoS Biol 4, e72 (2006). These results suggest that chromosomes with the C-14010 mutation have rapidly risen to high frequency in East African populations due to strong positive selection, consistent with a functional role of this mutation.

Lastly, analyses of transcriptional regulation of the LCT promoter in vitro indicate that otherwise identical constructs with a C-14010 allele consistently produced ˜18% more luciferase than constructs with the G-14010 allele (FIG. 5), an increase in transcription similar to that observed for the T-13910 allele in Europeans. Olds et al., Lactase persistence DNA variant enhances lactase promoter activity in vitro: functional role as a cis regulatory element, Hum Mol Genet 12, 2333-40 (2003); Troelsen et al., An upstream polymorphism associated with lactase persistence has increased enhancer activity, Gastroenterology 125, 1686-94 (2003).

Furthermore, two additional mutations, G-13907 and G-13915, have been identified at ≧5% frequency in the Beja from Sudan and Northern Kenyans, that are on haplotype backgrounds that increases gene expression by ˜18-30% compared to the ancestral haplotypes. (FIG. 4). Although SNPs T/G-13915 and C/G-13907 are associated with a mean rise in blood glucose of 3.18 and 3.99 mM/L in heterozygotes, respectively (FIG. 2b), these associations were less significant in the subpopulations or in the meta-analysis (FIG. 3), possibly due to small sample size and loss of power for these SNPs. Additionally, chromosomes with the G-13907 and G-13915 mutations exhibit EHH spanning ˜1.4 Mbp and ˜1.1 Mbp, respectively (FIG. 9). These results indicate that G-13915 and G-13907 are likely candidates to be LCT regulatory mutations. Accordingly, as discussed herein, these SNPs remain important for the methods, genotyping and kits detailed herein. Identification of transcription factors that bind to the sites of the C-14010, T-13915, and G-13907 mutations would also be informative for clarifying the possible role of these mutations in regulating LCT gene expression.

Adaptive Significance of LP and Implications for the Origins of Pastoralism in Africa

In connection with the various exemplary embodiments of the present invention, the adaptive significance of LP and implications for the origins of pastoralism in Africa are investigated and discussed.

For example, archeological evidence suggests that cattle domestication originated in Southern Egypt as early as ˜9 kya, but no later than ˜7.7 kya, and in the Middle East ˜7-8 kya, consistent with the age estimate of ˜8-9 kya for the T-13910 mutation in Europeans. Gifford-Gonzalez, in African Archeology (ed. Stahl, A. B.) pp. 187-224 (Blackwell Publishing, London, 2005). The estimated age of the C-14010 mutation in African populations, ˜2.7-6.8 kya (95% CI ˜1.2-23 kya), is consistent with archeological data indicating that pastoralism did not spread south of the Sahara and into N. Kenya until ˜4.5 kya, and into S. Kenya and N. Tanzania until ˜3.3 kya. Gifford-Gonzalez, in African Archeology (ed. Stahl, A. B.) pp. 187-224 (Blackwell Publishing, London, 2005); Ambrose, Chronology of the Later Stone Age and food production in East Africa, J. Arch Sci 25, 377-391 (1998). The ability to digest milk as adults was likely to be adaptive due to the increased nutritional benefits from milk (carbohydrates as well as fat, protein, and calcium), but also because milk is an important source of water in arid regions. Considering the symptoms of lactose intolerance, which includes water loss from diarrhea, individuals who had the LP-associated mutations and could tolerate milk could have had a very strong selective advantage. This is supported by our high estimates for the selection coefficient (s=0.035-0.097). Because the selective force, adult milk consumption, is associated with the cultural development of cattle domestication, the recent and rapid spread of the LP-associated mutations, together with the practice of pastoralism in East Africa, is an excellent example of ongoing adaptation in humans and gene-culture co-evolution.

It is observed that the oldest age estimates of the C-14010 mutation, ˜6-7 kya [95% CI ˜2-16 kya], in the Kenya NS and Tanzania AA populations (also observed is an old age estimate in the Tanzanian Sandawe; however, it's low frequency suggests it was introduced via recent gene flow) (Table 1). However, it is not distinguished with certainty whether this mutation first arose in the Cushitic-speaking AA populations, who are thought to have migrated into Kenya and Tanzania from Ethiopia ˜5 kya and practice a mixture of agriculture and pastoralism, or in the nilotic-speaking NS populations, who are thought to have migrated into Kenya and Tanzania from Southern Sudan within the past ˜3,000 years and are strict pastoralists. Newman, The Peopling of Africa (Yale University Press, New Haven and London, 1995); Gifford-Gonzalez, in African Archeology (ed. Stahl, A. B.) pp. 187-224 (Blackwell Publishing, London, 2005). These results are consistent with both linguistic and genetic data (Reed and Tishkoff, unpublished data) indicating cultural exchange and genetic admixture between these groups. Ehret, in Culture History in the Southern Sudan, pp. 19-48. Memoire 8. Nairobi: (eds. Mack, J. & Robertshaw, P.) (British Institute in Eastern Africa, 1983). The absence of C-14010 in the Southern Sudanese NS-speaking populations suggests that this mutation either originated in or was introduced to the Kenyan NS populations subsequent to their migration from Southern Sudan. Regardless of the population origins of the C-14010 mutation, it rapidly spread, together with the cultural practice of pastoralism, throughout the region, consistent with a demic diffusion model of cultural and population expansion. Cavalli-Sforza et al., History and Geography of Human Genes (Princeton University Press, Princeton, 1994).

Implications for Identifying Disease-Risk Mutations

In connection with the various exemplary embodiments of the present invention, the implications for identifying disease-risk mutations are investigated and discussed.

For example, it has been hypothesized that genetic mutations associated with both Mendelian (e.g. sickle cell anemia, G6PD deficiency) and common complex diseases (e.g. hypertension, diabetes, obesity, asthma) may be at high frequency in modern populations because they were adaptive in ancient environments. The International HapMap Consortium, A haplotype map of the human genome. Nature 437, 1299-1320 (2005); Voight et al., A map of recent positive selection in the human genome, PLoS Biol 4, e 72 (2006); Tishkoff et al., Patterns of human genetic diversity: implications for human evolutionary history and disease, Annu Rev Genomics Hum Genet 4, 293-340 (2003), Di Rienzo et al., An evolutionary framework for common diseases: the ancestral-susceptibility model, Trends Genet 21, 596-601 (2005); Tishkoff et al., Haplotype diversity and linkage disequilibrium at human G6PD: recent origin of alleles that confer malarial resistance, Science 293, 455-62 (2001). Thus, identification of loci which are targets of natural selection could be informative for identifying disease-risk alleles. The rapid increase in frequency of geographically restricted LP-associated mutations is an example of local adaptation that would have been missed by studying other African populations, such as the Yoruba, which did not show a signature of selection at LCT in the HapMap dataset. The International HapMap Consortium, A haplotype map of the human genome. Nature 437, 1299-1320 (2005); Voight et al., A map of recent positive selection in the human genome, PLoS Biol 4, e72 (2006). Because of the possibility that disease-associated mutations may also be geographically restricted due to recent, local adaptation, these results suggest the importance of resequencing analyses in multiple populations, even from within one geographic region such as Africa.

The studies herein also indicate how challenging it may be to identify alleles that are targets of selection. Networks of the 98 kb region encompassing the LCT and MCM6 genes (FIG. 4) indicates several haplotypes that are at high frequency in global populations and have ancestral alleles at the LP-associated SNPs (i.e. haplotypes D and E; FIG. 4). Based on a single factor ANOVA test, neither of these haplotypes is significantly associated with the LP phenotype (P=0.20 and P=0.058, respectively). The only difference between LP-associated haplotype F and the ancestral haplotype E, is the single G to C mutation at position −14010. The presence of these globally common haplotypes that are identical over at least 98 kb raises the possibility that there have been additional selective sweeps in the LCT/MCM6 gene region, possibly unrelated to LCT gene expression and confounding the haplotype based inference of selection at LCT.

Convergent Evolution of LP-Associated Mutations in Europeans and Africans

In connection with the various exemplary embodiments of the present invention, the convergent evolution of LP-associated mutations in Europeans and Africans has been investigated and discussed.

For example, these data suggest that at least two, and probably four or more distinct mutations associated with LP have evolved independently in European and African populations due to convergent evolution in response to a strong selective force, adult milk consumption: T-13910 in Europeans and C-14010, G-13907, and G-13915 in Africans. These mutations arose on highly divergent haplotype backgrounds which are geographically restricted (FIG. 4b), but they do not account for all of the phenotypic variation, particularly in the NS Sudanese and Hadza. (FIG. 2). Therefore, it is likely that there are additional LP-associated mutations in Africans.

Surprisingly, the Hadza population of Tanzania, who speak a click-language and subsist by hunting and gathering, have the LP phenotype at ˜50% frequency (FIG. 2a), suggesting that either the Hadza descend from a pastoralist population or that the LP trait may be adaptive for something other than milk digestion. These results, which should be confirmed in a larger sample, add to the mystery of the origins of the Hadza and their relationship to other click-speaking populations in Africa.

In conclusion, multiple independent mutations have allowed various human populations to quickly modify LCT expression and have been strongly adaptive in adult milk-consuming populations, emphasizing the importance of regulatory mutations in recent human evolution. Further resequencing and genotype/phenotype analyses in Africa, particularly in populations that lack the C-14010 mutation, will allow for further identifying additional LP-associated mutations. Once these mutations are identified, genotype analyses in a broader set of African populations will be informative for reconstructing an even more complete history of adaptation to pastoralism in Africa.

### EXAMPLES

By way of example, without limitation, exemplary embodiments of the present invention may also be illustrated with reference to the examples. Accordingly, in accordance with the exemplary embodiments of the present invention, the following is provided.

DNA samples. Tanzanian DNA samples were collected from individuals residing in the Arusha and Dodoma provinces of Tanzania. Kenyan samples were collected in the Rift valley, Nyanza, and Eastern provinces of Kenya. Sudanese samples were collected in the Khartoum and Kasala provinces of the Sudan. Samples were grouped according to self-identified ethnicity from unrelated individuals. Ethnic groups, number of individuals sampled, language classification, and subsistence classification are given in Table 2. White cells were isolated in the field from whole blood using a salting out procedure modified from Miller et al. and DNA was extracted in the lab using a Purgene DNA extraction kit (Gentra). Miller et al., A simple salting out procedure for extracting DNA from human nucleated cells, Nucleic Acids Res 16, 1215 (1988).

Phenotype Test. The Lactose Tolerance Test (LTT) measures rise in blood glucose levels following consumption of 50 g of lactose (equivalent to ˜1-2 liters of cow's milk). Arola, Diagnosis of hypolactasia and lactose malabsorption, Scand J Gastroenterol Suppl 202, 26-35 (1994). Baseline glucose levels were measured by obtaining blood via a fingerprick and using an Accucheck Advantage glucose monitor and Accucheck Comfort Strips (Roche). Blood glucose levels were obtained 20, 40, and 60 minutes after consumption of 50 g of lactose (Quintron) dissolved in 250 ml water. Based on manufacturer recommendation, glucose values were adjusted based on previously determined error associated with use of the Comfort Strip Curves according to the following regression equation: y=0.985x−7.5, where x is the measured glucose value. The maximum rise in glucose level compared to baseline values was determined. We used the following definition to classify individuals as “Lactase Persistent”: a rise of >1.7 mM/L was classified as “Lactase Persistent”, a rise of <1.1 mM/L was classified as “Lactase Non-Persistent”, a rise of 1.1-1.7 is ambiguous and classified as “Lactase Intermediate Persistent”. Arola, H., Diagnosis of hypolactasia and lactose malabsorption, Scand J Gastroenterol Suppl 202, 26-35 (1994). It should be noted that there is likely to be some error in phenotype classification due to administering the test under field conditions. The LTT test is less reliable than determining lactase enzyme activity directly by intestinal biopsy, with a false negative rate (i.e. LP individuals may be misclassified as LNP) as high as 23-30%. Hollox et al., in The Genetic Basis of Common Diseases (eds. King, R. A., Rotter, J I. & Motulsky, A. G.) 250-265 (Oxford University Press, Oxford, 2002); Arola, Diagnosis of hypolactasia and lactose malabsorption, Scand J Gastroenterol Suppl 202, 26-35 (1994). Although more accurate indirect tests exist (i.e. determination of urinary galactose after inclusion of ethanol with the lactose load or a hydrogen breath test), these were not feasible to do in remote locations in Africa. Arola, Diagnosis of hypolactasia and lactose malabsorption, Scand J Gastroenterol Suppl 202, 26-35 (1994). In addition, it was not possible to ensure that participants had fasted for at least 8 hours prior to administration of the test, as recommended in clinical settings, although most participants indicated that they had not eaten for at least several hours prior to testing. Hollox et al., in The Genetic Basis of Common Diseases (eds. King, R. A., Rotter, J. I. & Motulsky, A. G.) 250-265 (Oxford University Press, Oxford, 2002).

Sequence Analysis. A 3,314 bp region encompassing intron 13 of MCM6 and a 1,761 bp region encompassing intron 9 was PCR amplified (FIG. 1 c, d) in 110 (69 LP and 40 LNP) individuals from Sudan (16 LP and 10 LNP), Kenya (36 LP and 17 LNP), and Tanzania (17 LP and 14 LNP) (primers and PCR conditions are discussed below). PCR products were prepared for sequencing with shrimp alkaline phosphatase and exonuclease I (U.S. Biochemicals). All nucleotide sequence data were obtained using the ABI Big Dye v3.1 terminator kit and 3730xl automated sequencer (Applied Biosystems). Sequence files were aligned and SNPs identified using the Sequencher v. 4.0.5 program (Gene Codes).

SNP genotyping. 146 SNPs were selected for genotyping from Bersaglieri et al., dbSNP, and the resequencing of introns 9 and 13 of MCM6 in the individuals listed above. Bersaglieri et al., Genetic signatures of strong recent positive selection at the lactase gene, Am J Hum Genet 74, 1111-20 (2004). All SNPs were genotyped in 494 samples. Following Bersaglieri et al., the SNPs were chosen to represent a large area on chromosome 2, but with increased density in the LPH and MCM6 gene regions (FIG. 1a). Bersaglieri et al., Genetic signatures of strong recent positive selection at the lactase gene, Am J Hum Genet 74, 1111-20 (2004). SNPs were also included that had previously been shown to be associated with LP in Europeans (C/T-13910 and G/A-22018) or appeared to be associated with LP based on the initial resequencing screen described above. SNP assays were designed with the SpectroDESIGNER software. SNP typing was performed with the Homogeneous Mass Extend assay (Sequenom) as described elsewhere. Whittaker et al., in Cell Biology: A laboratory Handbook (ed. Celis, J) (Elsevier, 2006). Genotyping was carried out at a multiplex level of up to 10 SNPs per well and data quality was assessed by duplicate DNAs (n=7 in triplicate). SNPs with more than one discrepant call or showing self priming in the negative control (water) were removed. Finally, removed were SNPs with call rates below 70%, and flagged markers that departed from Hardy Weinberg equilibrium (p<0.001). A total of 123 SNPs, of which seven were monomorphic, passed quality control, including 79 SNPs from Bersaglieri et al. (Bersaglieri et al., Genetic signatures of strong recent positive selection at the lactase gene, Am J Hum Genet 74, 1111-20 (2004)), 34 SNPs from dbSNP, and 9 SNPs from resequencing (5 and 5 from inton 9 and 13, respectively) were included in the final analysis. See Table 3.

Genotype/Phenotype association tests. Genotype/phenotype association for data binned into LP, LNP, and LIP classifications was determined by a chi-square test. The degrees of freedom for the chi-square test are calculated as the product of the number of phenotypes minus one and the number of genotypes minus one. In cases where there were low expected cell counts (<5), cells were pooled to satisfy Cochran's guidelines. Cochran, W. G., Some methods for strengthening the common chi-square test, Biometrics 10, 417-451 (1954). Because the phenotype (rise in blood glucose) is a continuous trait, we also used a least-squares linear regression approach to test for significant genotype/phenotype associations. Cheung et al., Mapping determinants of human gene expression by regional and genome-wide association, Nature 437, 1365-9 (2005). This method avoids the loss of information that may arise from binning the phenotype into discrete categories. For each SNP, different homozygotes were assigned to values of 0 or 1 and heterozygotes were assigned an intermediate genotype value of ½ (i.e. assumes an additive model). Next, a linear regression was fit to the x-axis genotype values and y-axis phenotypes (glucose rise). The resulting r² and P-values were recorded as measures of the degree of association. Because of the large amount of multiple testing (123 SNPs), a significant association was determined after applying a conservative Bonferroni P-value correction.

Combined population meta-analysis. In order to both gain statistical power and to avoid the issues of population stratification, we conducted a meta-analysis on the results of the association tests in the individual geographic-linguistic populations. This was done by combining the P-values for each SNP over k populations in an unweighted Z-transform test according to the following equation:

$${Z}_{meta} = \frac{{\sum }_{i = 1}^{k} {Z}_{i}}{\sqrt{k}}$$

where Zᵢ is the Z-score of the standard normal curve corresponding to the P-value from an individual population phenotype-genotype regression and Zₘₑₜₐ is the Z-score for the combined meta-analysis. Stouffer et al., The American Soldier, Vol. 1: Adjustment during Army Life (Princeton University Press, Princeton, 1949). This method tests for a skew in the overall distribution of P-values (from tests in individual populations) regardless of the significance of any individual test and allows us to regain some of the power that was lost by dividing the data into smaller groups

ANOVA analyses. A single factor ANOVA was used to test for a significant difference in phenotypes between the two common haplotypes (D and E) in the LCT-MCM6 region (FIG. 4a) and all other haplotypes, after individuals carrying a C-14010 and/or a G-13907 and/or a G-13915 allele (or unknown genotypes at any of these three markers) had been removed. An ANOVA was also used to quantify the overall variation in phenotype measures explained by G/C-14010, T/G-13915, and C/G-13907; each of the 10 compound genotypes found in the dataset were treated as a category.

Homozygosity plots. To visualize the extent of homozygosity on chromosomes with the LP associated alleles, individuals that are homozygous for the ancestral and derived alleles at G/C-14010 and C/T-13910 SNPs were selected and the extent of continuous homozygosity at each assayed SNP, in each direction, was plotted. Note that this is the actual measured homozygosity and, thus, is independent of haplotype phase estimation but is sensitive to inbreeding.

Haplotype phase estimation. fastPHASE was used, with population label information, in order to estimate phased haplotype backgrounds. Scheet et al., A fast and flexible statistical model for large-scale population genotype data: applications to inferring missing genotypes and haplotypic phase, Am J Hum Genet 78, 629-44 (2006).

Calculation of iHS scores. We calculated iHS scores as per Voight et al. for each subpopulation for all SNPs in the region. Voight et al., A map of recent positive selection in the human genome, PLoS Biol 4, e72 (2006). In calculating the scores, we used an interpolated recombination map estimated from the Hapmap project Yoruba dataset. The International HapMap Consortium, A haplotype map of the human genome, Nature 437, 1299-1320 (2005). iHS scores were standardized using estimates of the mean and standard deviation obtained via coalescent simulation under a variety of demographic models. These simulations were tailored to match the frequency spectrum, SNP density, and recombination profile of the observed data. Alternative demographic models included either exponential growth or a bottleneck (which varied in onset, severity, duration, and population size recovery after the bottleneck). 1000 repetitions of each demographic model were simulated, and the distribution of iHS scores for sites matching the frequency (within 2.5%) as well as position of C-14010 were calculated. Empirical p-values which count the number of simulated iHS scores for each demographic model that exceeded (i.e. were more negative) than the observed iHS statistic, as well as a description of the models (and results), are presented in Table 4. In addition, iHS scores were standardized empirically by comparison with the Yoruba hapmap data for alleles at the same frequency as C-14010.

Estimating selection intensity and sweep ages. We applied a rejection-sampling approach using the centiMorgan (cM) span surrounding the selected site to estimate selection intensity and ages of the candidate LP-associated mutations for each population. Pritchard et al., Population growth of human Y chromosomes: a study of Y chromosome microsatellites, Molecular Biology and Evolution 16, 1791-1798 (1999). Point estimates for the selection intensity and ages are presented, assuming an additive or fully dominant fitness effect. Although our model assumes constant population size, previous studies have demonstrated that for an allele that rapidly increases in frequency, population demographic history has only a modest effect on allele age estimates. Tishkoff et al., Haplotype diversity and linkage disequilibrium at human G6PD: recent origin of alleles that confer malarial resistance, Science 293, 455-62 (2001); Wiuf, Recombination in human mitochondrial DNA?, Genetics 159, 749-56 (2001).

Due to the way that SNPs were ascertained, the allele frequency spectrum departs from the expectation for DNA sequence data. To model the effect of ascertainment bias of SNPs selected for genotyping, we followed the approach in Voight et al. Voight et al., A map of recent positive selection in the human genome, PLoS Biol 4, e72 (2006). In addition, the observed data vary in terms of SNP density: a dense central core region flanked by regions with lower SNP density (on average). To match this feature of the data, a secondary rejection step was applied such that the average SNP density for central and flanking regions (both left and right) matched the observed density. With respect to recombination, for each simulation we chose to exactly match the recombination map estimated from the data using the Li and Stephens algorithm. Li et al., Modeling linkage disequilibrium and identifying recombination hotspots using single-nucleotide polymorphism data, Genetics 165, 2213-33 (2003). For all populations, we calculated cM spans assuming the estimated population genetic map for the Yoruba Hapmap dataset (The International HapMap Consortium, A haplotype map of the human genome, Nature 437, 1299-1320 (2005)), and calculated those distances assuming the rates estimated from the deCODE genetic map across 40 Mb flanking this region on chromosome 2. Kong et al., A high-resolution recombination map of the human genome, Nat Genet 31, 241-7 (2002).

Phylogenetic analsyses. Haplotype networks were generated using the median-joining algorithm of Network 4.1.1.1 for SNPs within the LPH and MCM6 gene regions from rs1042712 to rs309125, spanning 98 kbp. Bandelt et al., Median-joining networks for inferring intraspecific phylogenies. Mol Biol Evol 16, 37-48 (1999). The root was inferred assuming the chimpanzee allelic state at each SNP is ancestral.

Vector construction, transfection and expression assay. The LCT “core” promoter, starting 3083 bp upstream of LCT at position −3 of the transcription start site, was PCR amplified using high-fidelity Phusion polymerase (Finnzyme, Espoo, Finland). PCR products were then cloned and ligated into a pGL3-Basic luciferase reporter (Promega, Madison, Wis., United States). Constructs including the 13^{th} intron on MCM6 were constructed by cloning 2035 bp, beginning at position −14,354 bp relative to LCT, 5′ of the “core” promoter. Caco-2 cells were then transfected with these constructs. 48 hours after transfection, wells were lysed and Luciferase activity was measured using the Dual-Luciferase Reporter Assay System (Promega) and a Veritas Microplate Luminometer (Turner BioSystems). Transfections of cells were performed six times for control and “core” promoters and 12 times for vectors with the intron from MCM6. The expression data was analyzed using paired t-tests.

In accordance with the exemplary embodiments described herein, the preferred primer sequences and annealing temperatures used for amplification of introns 9 and 13 of MCM 6 may be described as follows.

### Methods

Primers sequences and annealing temperatures used
for PCR amplification of introns 9 and 13 of MCM6.

| | | |
|---|---|---|
| MCM6 Intron 9 Primers | Sequence | Annealing Temperature |

| | | |
|---|---|---|
| MCM6-9-forward | CCGAGGGAGAGAAACCTTC | 61.6° |
| MCM6-9-reverse | TCAACAAGGCTATGGACGATG | |

| MCM6 Intron 13 Primers | Sequence | Annealing Temperature |
|---|---|---|
| First fragment | | |

| | | |
|---|---|---|
| MCM6-13-forward | ATCTCCGCCAGAGAGATGG | 61.6° |
| MCM6-13seq3-reverse | TCATAGATGTTTTCAATTCTTCAAGT | |
| Second fragment | | |

| | | |
|---|---|---|
| MCM6-13seq4-forward | GGATCTCCTTTTGGACTTTCC | 61.6° |
| MCM6-13seq6-reverse | TGGACCTAAACCAATAATGATGAA | |

Primer sequences used to sequence introns 9 and 13 of MCM6

| | |
|---|---|
| MCM6 Intron 9 Primers | Sequence |

| | |
|---|---|
| MCM6-9seq1-forward | ACCAGTGGTAAAGCGTCCAG |
| MCM6-9seq1-reverse | AACAGCAAACACACGTGCTC |
| MCM6-9seq2-forward | TGCATTGAGCCAAGATTGTG |
| MCM6-9seq2-reverse | TAGCCAGGTGTGGTGGTGTG |
| MCM6-9seq3-forward | TCCCTGTGGTAGCAGACTTTG |
| MCM6-9seq3-reverse | TCCCGCACGTCCATCTTATC |

| | |
|---|---|
| MCM6 Intron 13 Primers | Sequence |

| | |
|---|---|
| MCM6-13seq1-forward | ATCTCCGCCAGAGAGATGG |
| MCM6-13seq1-reverse | GCTTTGGTTGAAGCGAAGAT |
| MCM6-13seq2-forward | GTTCTTTGAGCCCTGCATTC |
| MCM6-13seq2-reverse | AGGTTCGGGGGTACACATGC |
| MCM6-13seq3-forward | AGATACCCTGGGACAAGGTC |
| MCM6-13seq3-reverse | TCATAGATGTTTTCAATTCTTCAAGT |
| MCM6-13seq4-forward | GGATCTCCTTTTGGACTTTCC |
| MCM6-13seq4-reverse | TTCAACAAGAAACACTGAAAAACA |
| MCM6-13seq5-forward | GTGAGCCATGTGCTTTCTCC |
| MCM6-13seq5-reverse | GCACGGTGGCTCATGTCTAT |
| MCM6-13seq6-forward | TCTTCTTTCTCAGCCTCCTG |
| MCM6-13seq6-reverse | TGGACCTAAACCAATAATGATGAA |

SEQUENCE ID NO. 1

GTAAGTTACCATTTAATACCTTTCATTCAGGAAAAATGTACTTAGACCCT
ACAATGTACTAGTAGGCCTCTGCGCTGGCAATACAGATAAGATAATGTAG
CCCC

SEQUENCE ID NO. 1 illustrates that “wild type” allele which includes: G-14010, T-13915 and C-13907, as measured from the start of the LCT gene.

SEQUENCE ID NO. 2

G/CTAAGTTACCATTTAATACCTTTCATTCAGGAAAAATGTACTTAGACC
CTACAATGTACTAGTAGGCCTCTGCGCTGGCAATACAGATAAGATAAT/G
GTAGCCCC/G

SEQUENCE ID NO. 2 is provided by illustrative purposes, to show both the “wild type” and “variant allele”, as indicated by G/C (G/C-14010), T/G (T/G-13915) and C/G (C/G-13907), as measured from the start of the LCT gene.

What has been described and illustrated herein are examples of the methods, tests, kits and/or nucleic acid molecules described herein along with some of their variations. The terms, descriptions and figures used herein are set forth by way of illustration only and are not meant as limitations. Those skilled in the art will recognize that many variations are possible within the spirit and scope of these examples, which intended to be defined by the following claims and their equivalents in which all terms are meant in their broadest reasonable sense unless otherwise indicated. Furthermore, various references are cited throughout the description herein, wherein the contents of each of these references are incorporated herein in their entirety.

What is claimed is:

1. A method for determining an individual's predisposition for lactase non-persistence, said method comprising:
determining the absence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene;
wherein the absence of the one or more single nucleotide polymorphisms indicates that the individual has a predisposition for lactase non-persistence

2. The method of claim 1, wherein the presence of at least one of the one or more single nucleotide polymorphisms indicates that the individual has a predisposition for lactase persistence.

3. The method of claim 1, wherein the one or more single nucleotide polymorphism comprises C-14010.

4. The method of claim 1, further comprising:
determining the absence of the one or more single nucleotide polymorphism by amplifying a nucleotide sequence of the gene associated with the expression of lactase-phlorizin hydrolase; and
detecting the absence of the single nucleotide polymorphism in the amplified nucleic acids.

5. The method of claim 4, where the step of detecting further comprises sequencing the amplified nucleotide sequence.

6. The method of claim 4, wherein the gene associated with the expression of lactase-phlorizin hydrolase is MCM 6.

7. The method of claim 1, wherein said method further comprises a test for lactose intolerance, wherein the absence of one or more of the single nucleotide polymorphisms indicates that the individual has a predisposition for lactose intolerance.

8. A method for determining an individual's predisposition for lactase persistence, said method comprising:
determining the presence of at least one variant allele having one or more single nucleotide polymorphisms within a gene associated with the expression of lactase-phlorizin hydrolase, wherein the single nucleotide polymorphism is selected from the group consisting essentially of C-14010, G-13915 and G-13907, as measured from the start of the LCT gene;
wherein the presence of the one or more single nucleotide polymorphisms indicates that the individual has a predisposition for lactase persistence.

9. The method of claim 8, wherein the presence of an allele of the gene associated with the expression of lactase-phlorizin hydrolase having at least one of G-14010, T-13915 and C-13907, as measured from the start of the LCT gene, indicates that the individual has a predisposition for lactase non-persistence as compared to the presence of one or more of the variant alleles having the single nucleotide polymorphism.

10. The method of claim 8, wherein the single nucleotide polymorphism is C-14010.

11. The method of claim 8, further comprising:
determining the presence of the single nucleotide polymorphism by amplifying a nucleotide sequence comprising the variant allele having the single nucleotide polymorphism selected from the group consisting essentially of C-14010, G-13915 and G-13907; and
detecting the presence of the single nucleotide polymorphism in the amplified nucleotide sequence.

12. The method of claim 11, where the step of detecting further comprises sequencing the amplified nucleic acids.

13. The method of claim 8, wherein the gene associated with the expression of lactase-phlorizin hydrolase is MCM 6.

14. The method of claim 8, wherein said method further comprises a test for lactose tolerance, wherein the presence of the one or more single nucleotide polymorphisms indicates that the individual has a predisposition for lactose tolerance.

15. A method for genotyping an individual comprising determining the absence or presence of a variant allele containing a single nucleotide polymorphism within a gene associated with the expression of lactase-phlorizin hydrolase, in a biological sample from the individual, wherein the single nucleotide polymorphism is selected from the group consisting essentially of C-14010, G-13915 and G-13907, measured from the start of the LCT gene.

16. The method of claim 15, wherein the absence of one or more of the single nucleotide polymorphisms indicates that the individual has a predisposition for lactase non-persistence as compared to the presence of one or more of the single nucleotide polymorphisms.

17. The method of claim 15, wherein the presence of one or more of the single nucleotide polymorphisms indicates that the individual has a predisposition for lactase persistence as compared to the absence of one or more of the single nucleotide polymorphisms.

18. The method of claim 15, wherein the single nucleotide polymorphism is C-14010.

19. The method of claim 15, further comprising:
determining the absence or presence of the single nucleotide polymorphism by amplifying a nucleotide sequence of the gene associated with the expression of lactase-phlorizin hydrolase; and
detecting the absence or presence of the single nucleotide polymorphism in the amplified nucleotide sequence.

20. The method of claim 19, where the step of detecting further comprises sequencing the amplified nucleotide sequence.

21. The method of claim 15, wherein the gene associated with the expression of lactase-phlorizin hydrolase is MCM 6.

22. An isolated nucleic acid molecule comprising a variant MCM 6 nucleotide sequence, a variant nucleotide sequence comprising intron 13 of MCM 6, or a sequence complementary thereto, wherein the said variant nucleotide sequence comprises at least a fragment of SEQ ID NO 1, wherein at least one of the following conditions applies:
a) the nucleotide at position 13907 is guanine;
b) the nucleotide at position 13915 is guanine; or
c) the nucleotide at position 14010 is cytosine.

23. The isolated nucleic acid molecule of claim 22, wherein said isolated nucleic acid molecule is located within a vector.

24. The isolated nucleic acid molecule of claim 23, wherein said vector is located within a transfected host cell.

25. The isolated nucleic acid molecule of claim 22, wherein said isolated nucleic acid molecule is included as part of a kit for determining an individual's predisposition for lactase persistence, lactase non-persistence, lactose tolerance or lactose intolerance.
